(19) United States
(12) Patent Application Publication
(10) Pub. No.: US 2024/0124851 A1
(43) Pub. Date: Apr. 18, 2024

(54) ESCHERICHIA COLI BACTERIOPHAGES AND USES THEREOF

(71) Applicant: Intralytix, Inc., Columbia, MD (US)

(72) Inventor: Alexander SULAKVELIDZE, Brookeville, MD (US)

(21) Appl. No.: 18/471,419

(22) Filed: Sep. 21, 2023

Related U.S. Application Data
(60) Provisional application No. 63/376,496, filed on Sep. 21, 2022.

(51) Int. Cl. C12N 7/00; A23L 3/3571; A61K 35/76; A61P 31/04

(52) U.S. Cl. CPC C12N 7/00; A23L 3/3571; A61K 35/76; A61P 31/04

(57) ABSTRACT

The present invention is directed to isolated bacteriophages having specificity and lytic activity against strains of pathogenic E. coli, methods of using the bacteriophages, progeny and derivatives derived therefrom, to control the growth of pathogenic E. coli in various settings (e.g., food safety, environmental—including food establishments and medical settings—sanitation, urinary tract infections, modulating microbiome, prebiotics, probiotics).

### CROSS-REFERENCE TO RELATED APPLICATIONS

The present application claims the benefit of U.S. Provisional Patent Application No. 63/376,496, filed Sep. 21, 2021, the disclosure of which is incorporated by reference in its entirety herein.

### INCORPORATION OF SEQUENCE LISTING

The sequence listing that is contained in the file named “041071_00115_Sequence_Listing.xml,” which is 1,124 kilobytes as measured in Windows 10 operating system and was created on Sep. 12, 2023, is filed electronically herewith and incorporated herein by reference.

### FIELD OF THE INVENTION

The present invention relates to eight novel bacteriophages designated (the “Deposited Bacteriophages”), and compositions and preparations corresponding thereto which possess lytic activity against strains of Escherichia coli capable of causing animal (including human) disease, including Shiga-toxin producing E. coli (STEC, also sometimes referred to as verocytotoxin-producing E. coli or VTEC), Enterohemorrhagic E. coli (EHEC), enteropathogenic E. coli (EPEC), enterotoxigenic E. coli (ETEC), enteroaggregative E. coli (EAEC), enteroinvasive E. coli (EIEC), diffusely adherent E. coli (DAEC), uropathogenic E. coli (UPEC), meningitis-associated E. coli (MNEC) (including neonatal meningitis-causing E. coli or NMEC), avian pathogenic E. coli (APEC), sepsis-associated E. coli (SAEC), mammary pathogenic E. coli, endometrial pathogenic E. coli, adherent invasive E. coli (AIEC), and necrotoxigenic E. coli (the “Targeted Bacteria” or “Pathogenic E. coli”).

### BACKGROUND OF THE INVENTION

### Bacteriophages

Bacteriophages are bacterial viruses that attach to their specific hosts and kill them by internal replication and bacterial lysis involving a complex lytic cycle involving several structural and regulatory genes. Phages are very specific in that they only attack their targeted bacterial hosts. They cannot infect human or other eukaryotic cells. Bacteriophages were first identified, in the early part of the 20th century, by Felix D'Herelle who called them bacteriophages or bacteria-eaters (from the Greek phago meaning to eat or devour) (Summers 1999)¹. It is estimated that there are approximately 10³¹ phage particles in the biosphere, and about 10²³ phage infections occur every second on a global scale (Suttle 2005), (Suttle 2007). ¹Full bibliographic citations are included at the end of the specification.

### Bacteriophage Structure

Although different bacteriophages may contain different materials, they all contain nucleic acid and protein. Depending upon the phage, the nucleic acid can be either DNA or RNA, and it can exist in various forms. The nucleic acids of phages often contain unusual or modified bases. These modified bases protect phage nucleic acid from nucleases that break down host nucleic acids during phage infection. The size of the nucleic acid varies depending upon the phage. The simplest phages only have enough nucleic acid to code for 3-5 average size gene products while the more complex phages may code for over 100 gene products (the largest bacteriophage genomes maybe >700 kb in size). The number of different kinds of protein and the amount of each kind of protein in the phage particle will vary depending upon the phage. The simplest phage has many copies of only one or two different proteins while more complex phages may have many different kinds. The proteins function in infection and to protect the nucleic acid from nucleases in the environment (McGrath and van Sinderen 2007).

The basic structural features of bacteriophages include their head or capsid, and tail. For example, T4, a common phage is among the largest phages; it is approximately 200 nm long and 80-100 nm wide. Most phages range in size from 24-200 nm in length.

All phages contain a head structure which can vary in size and shape. Some are icosahedral (20 sides) others are filamentous. The head or capsid is composed of many copies of one or more different proteins. Inside the head is found the nucleic acid. The head acts as the protective covering for the nucleic acid. Many but not all phages have tails attached to the phage head. The tail is a hollow tube through which the nucleic acid passes during infection. The size of the tail can vary, and some phages do not even have a tail structure. In the more complex phages like T4 the tail is surrounded by a contractile sheath which contracts during infection of the bacterium. At the end of the tail, the more complex phages like T4 have a base plate and one or more tail fibers attached to it. The base plate and tail fibers are involved in the binding of the phage to the bacterial cell. Not all phages have base plates and tail fibers. In these instances, other structures are involved in binding of the phage particle to the bacterium (Kutter and Sulakvelidze 2005).

### Bacteriophages Infect Bacteria

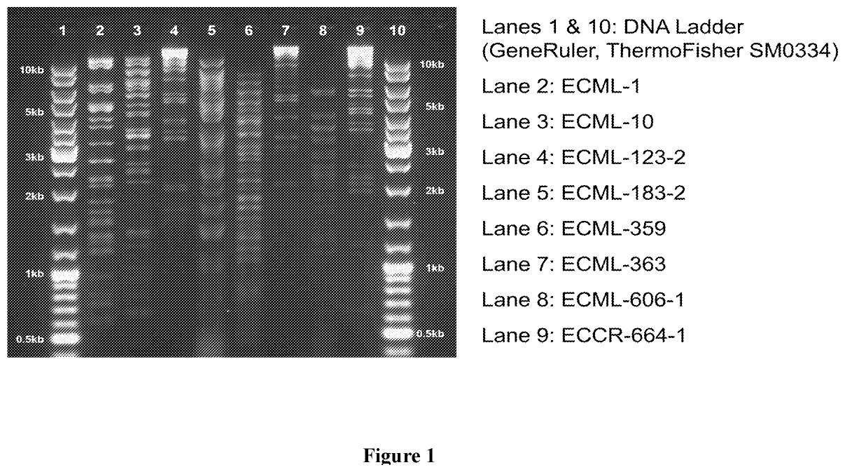

Replication cycle of a lytic bacteriophage is described in detail in Sulakvelidze 2011, especially in FIG. 1 incorporated herein by reference. The first step in the lytic cycle process is the adsorption of the phage to the bacterial cell. This step is mediated by the tail fibers or by some analogous structure on those phages that lack tail fibers, and it is reversible. The tail fibers attach to specific receptors on the bacterial cell, and the host specificity of the phage (i.e., the bacteria that it can infect) is usually determined by the type of tail fibers that a phage has. The nature of the bacterial receptor varies for different bacteria (e.g., proteins on the outer surface of the bacterium, LPS, pili, and lipoprotein). These receptors are on the bacteria for other purposes, and phage have evolved to use these receptors for infection (Kutter and Sulakvelidze 2005). The attachment of the phage to the bacterium via the tail fibers is a weak one and is reversible. Irreversible binding of phage to a bacterium is mediated by one or more of the components of the base plate. Phages lacking base plates have other ways of becoming tightly bound to the bacterial cell. The irreversible binding of the phage to the bacterium results in the contraction of the sheath (for those phages which have a sheath), and the hollow tail fiber is pushed through the bacterial envelope. Phages that do not have contractile sheaths use other mechanisms to get the phage particle through the bacterial envelope. Some phages have enzymes that digest various components of the bacterial envelope (McGrath and van Sinderen 2007).

### Lytic (Virulent) Phage Life Cycle

Lytic or virulent phages are phages which can only multiply on bacteria and kill the cell by lysis at the end of the life cycle. During the eclipse phase, no infectious phage particles can be found either inside or outside the bacterial cell. The phage nucleic acid takes over the host biosynthetic machinery, and phage specified mRNAs and proteins are made. There is an orderly expression of phage directed macromolecular synthesis, just as one sees in animal virus infections. Early mRNAs code for early proteins that are needed for phage DNA synthesis and for shutting off host DNA, RNA, and protein biosynthesis. After phage DNA is made, late mRNAs and late proteins are made. The late proteins are the structural proteins that comprise the phage as well as the proteins needed for lysis of the bacterial cell (McGrath and van Sinderen 2007). In the Intracellular Accumulation Phase, the nucleic acid and structural proteins that have been made are assembled and infectious phage particles accumulate within the cell. During the Lysis and Release Phase, the bacteria begin to lyse due to the accumulation of the phage lysis protein (e.g., lysin), and intracellular phage are released. The number of particles released per infected bacteria is typically 10-250 but may be as high as 1,000.

A common assay for lytic phage is the plaque assay where lytic phages are enumerated by a plaque assay. A plaque is a clear area which results from the lysis of bacteria. Each plaque arises from a single infectious phage. The infectious particle that gives rise to a plaque is called a PFU (plaque forming unit) (Adams 1959), (Kutter and Sulakvelidze 2005).

### Temperate (“Lysogenic”) Phage Life Cycle

Temperate phages (sometimes also mistakenly called “lysogenic” phages) are those that can either multiply via the lytic cycle or enter a quiescent state in the cell. In this quiescent state most of the phage genes are not transcribed; the phage genome exists in a repressed state. The phage DNA in this repressed state is called a prophage because it is not a phage per se, but it has the potential to produce phage. In most cases the phage DNA integrates into the host chromosome and is replicated along with the host chromosome and passed on to the daughter cells. The cell harboring a prophage is not adversely affected by the presence of the prophage, and the lysogenic state may persist indefinitely. The cell harboring a prophage is termed a lysogen. See (McGrath and van Sinderen 2007), herein incorporated by reference in its entirety.

Anytime a lysogenic bacterium is exposed to adverse conditions, the lysogenic state can be terminated. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. See (Kutter and Sulakvelidze 2005), herein incorporated by reference in its entirety.

### Lytic and Lysogenic Bacteriophages

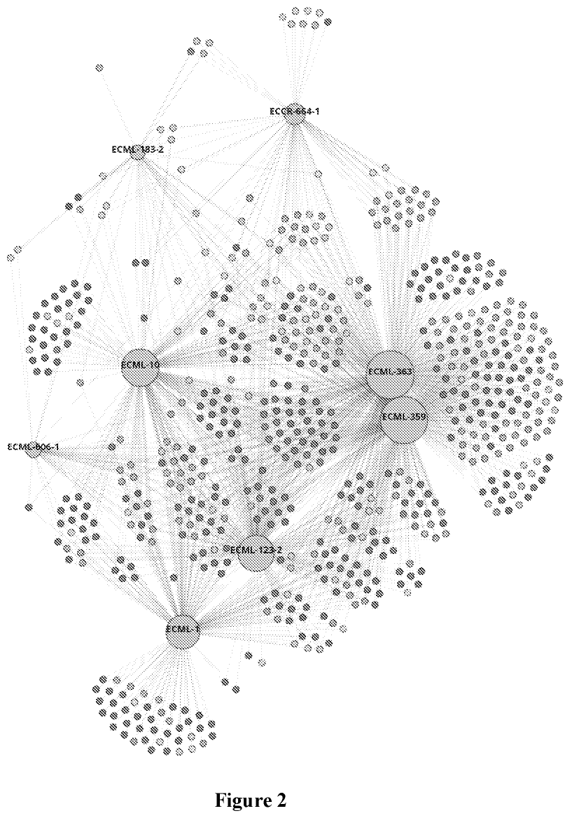

Bacteriophages have a lytic cycle or a lysogenic cycle, but some bacteriophages can carry out both. Differences in the replication cycles of lytic and temperate phages are schematically described in detail in Sulakvelidze 2011, especially in FIG. 2 incorporated herein by reference. With lytic phages such as the T4 phage, bacterial cells are broken open (lysed) and destroyed after immediate replication of the virion. As soon as the cell is destroyed, the new bacteriophage viruses can find new hosts (Kutter and Sulakvelidze 2005). In contrast, the lysogenic cycle does not result in immediate lysing of the host cell. Those phages able to undergo lysogeny are known as temperate phages. Their genome will integrate with host DNA and replicate along with it harmlessly or may even become established as a plasmid or prophage. The virus remains dormant until host conditions are stressed or deteriorate (e.g., due to depletion of nutrients, or exposure to UV light) then the endogenous phages (known as prophages) become active. At this point they initiate the reproductive cycle resulting in lysis of the host cell. As the lysogenic cycle allows the host cell to continue to survive and reproduce, the virus is reproduced in the host cell's offspring. See (Kutter and Sulakvelidze 2005).

E. coli Bacteria

Escherichia coli (E. coli) is a Gram-negative, facultative anaerobic, rod-shaped, coliform bacterium of the genus Escherichia (named after its discoverer Theodor Escherich), family Enterobacteriaceae, order Enterobacteriales, class Gammaproteobacteria, phylum Proteobacteria. Many E. coli strains are normal commensal of a healthy human gastrointestinal tract (E. coli typically colonizes the gastrointestinal tract of human infants within a few hours after birth), and those strains are non-pathogenic for healthy, immunocompetent humans (Kaper, Nataro et al. 2004). However, some E. coli strains can and do cause illness even in healthy people as well as in pets and in agriculturally important animals such as pigs or poultry. These disease-causing E. coli include Shiga-toxin producing E. coli (STEC, also sometimes referred to as verocytotoxin-producing E. coli or VTEC) (and their subset enterohemorrhagic E. coli or EHEC), enteropathogenic E. coli (EPEC), enterotoxigenic E. coli (ETEC), enteroaggregative E. coli (EAEC), enteroinvasive E. coli (EIEC) diffusely adherent E. coli (DAEC), uropathogenic E. coli (UPEC), meningitis-associated E. coli (MNEC) (including neonatal meningitis-causing E. coli or NMEC), avian pathogenic E. coli (APEC), sepsis-associated E. coli (SAEC), mammary pathogenic E. coli, endometrial pathogenic E. coli, adherent invasive E. coli (AIEC), and necrotoxigenic E. coli (Kaper, Nataro et al. 2004), (Etcheverria, Lucchesi et al. 2016).

The “pathogenic E. coli strains” as defined herein are STEC (VTEC), EHEC, EPEC, ETEC, EAEC, EIEC, DAEC, UPEC, MNEC (including NMEC), APEC, AIEC, sepsis-associated E. coli, mammary pathogenic E. coli, endometrial pathogenic E. coli, necrotoxigenic E. coli, or a combination thereof. Pathogenic E. coli developed an impressive array of virulence markers that enable them to trigger animal (including human) illness. Some of the virulence factors are summarized in Kaper, Nataro et al. 2004, which is herein incorporated by reference in its entirety. Some example virulence factors include colonization factors, fitness factors, toxins, and effectors. The diseases caused by pathogenic E. coli can be grouped into two main categories: diseases of humans and diseases of animals. Below we briefly discuss both.

### Diseases in Humans

Arguably the best-known human “pathogenic E. coli” serotype is E. coli O157:H7. This serotype is one of the seven serotypes currently included in the Shiga toxin-producing E. coli (STEC) group (also sometimes referred to as Verocytotoxin-producing E. coli or VTEC) which also includes enterohemorrhagic E. coli or EHEC. It is a major foodborne bacterial pathogen, which was first associated with human illness during an outbreak of hemorrhagic colitis in 1982, and has been listed by the Centers for Disease Control and Prevention as a national notifiable disease since 1994 (Bell, Goldoft et al. 1994), (Scallan, Hoekstra et al. 2011). This bacterium has been estimated to cause >63,000 foodborne illnesses and approximately 61 deaths annually in the United States (Bell, Goldoft et al. 1994), (Rangel, Sparling et al. 2005). E. coli O157:H7 infections are of particular concern in young children and elderly persons because it is associated with hemolytic uremic syndrome which may permanently damage the kidneys (Buzby 2001).

In addition to being significant to public health, the economic impact of STEC E. coli contamination of foods is substantial. It has been estimated that hospitalizations and deaths due to E. coli O157:H7 infections in the United States may lead to $405 million in medical cost and lost productivity annually (Frenzen, Drake et al. 2005). Furthermore, substantive costs to manufacturers and growers may be incurred in the form of product loss and brand-damaging publicity associated with recalling products contaminated with this bacterium. These costs significantly increase (due to additional legal fees and settlement agreements) if the consumption of those foods results in human illness or mortality and may force the company out of business (Buzby and Roberts 1997), (Buzby and Roberts 2009), (Pennington 2010). For example, a single E. coli O157:H7 outbreak associated with contaminated spinach in 2006 cost the spinach industry between $37 and $74 million. Thus, there are very strong public health and economic incentives to develop novel, environmentally friendly, safe, and effective approaches for managing STEC contamination of a broad range of foods.

Among other “pathogenic E. coli,” enterotoxigenic Escherichia coli or ETEC are also an important cause of bacterial diarrheal illness, e.g., infection with ETEC is the leading cause of travelers' diarrhea and a major cause of diarrheal disease in lower-income countries, especially among children. Another example of pathogenic E. coli of serious public health concern is enteroinvasive E. coli (EIEC); these bacteria cause a syndrome that is identical to shigellosis, with profuse diarrhea and high fever. Overall, the pathogenic E. coli infections are characterized with diarrhea, vomiting, abdominal pain, and fever—and in some cases serious complications or even death (e.g., STEC E. coli can cause potentially fatal hemolytic uremic syndrome; EPEC remains an important cause of fatal infant diarrhea in developing countries, etc.). Also, meningitis-associated E. coli (MNEC) (including neonatal meningitis-causing E. coli or NMEC)—and especially strains with K1 capsular polysaccharide antigen—remain the most common Gram-negative bacillary organism that cause meningitis (Kim 2016). Adherent-invasive E. coli (AIEC) have been implicated in the pathogenesis of inflammatory bowel disease (IBD), including Crohn's disease (Prudent, Demarre et al. 2021). Another group of pathogenic E. coli, UPEC, are a major cause of urinary tract infections (UTIs). The UTI infections are widespread: about 150 million people worldwide develop UTI each year. Also, it is estimated that 40% of women develop at least one UTI during their lifetime and that 11% of women over 18 years have at least one episode of UTI per year. With roughly eleven-million cases reported in the U.S. alone each year, the costs of UTI infections are estimated $5 billion annually (Terlizzi, Gribaudo et al. 2017). For a review of some of the pathogenic E. coli, See (Kaper, Nataro et al. 2004), herein incorporated by reference in its entirety.

### Diseases in Animals

Many animals carry E. coli—including pathogenic E. coli—as part of their normal flora. In many instances, such carriage does not cause disease in animals but does present significant public health concern because these E. coli strains can spread from cows, goats, sheep, deer, and other animals to humans where they can trigger various diseases, some of them potentially fatal. However, pathogenic E. coli can and do cause diseases in animals—and some examples are briefly discussed below.

Various serotypes of enterotoxigenic E. coli (ETEC) can cause either diarrhea or septicemiain young calves (usually during the first 4 days of life). Two main E. coli pathotypes are involved in enteric colibacillosis: enterotoxigenic E. coli (ETEC) and enteropathogenic E. coli (EPEC), with ETEC being the most important pathotype in swine. The ETEC causing neonatal colibacillosis mostly carry the fimbriae F4 (k88), F5 (k99), F6 (987P) or F41, while the ETEC of post-weaning diarrhea carry the fimbriae F4 (k88) and F18. These fimbriae adhere to specific receptors on porcine intestinal brush border epithelial cells (enterocytes), starting the process of enteric infection (Luppi 2017). VTEC (STEC) also can cause edema disease in pigs, usually after weaning and between the age of 4 and 12 weeks. Infection with ETEC harboring the F4 (K88) fimbriae (ETEC F4) is one of the most important causes of postweaning diarrhea in pigs. As noted above, this pathotype is characterized by the expression of an F4 fimbrial adhesin which induces bacterial attachment to specific F4 receptors located in the brush border of the swine intestine and secretion of enterotoxins that cause diarrhea (Fairbrother, Nadeau et al. 2005).

In many avian animals, including chickens, pathogenic E. coli can cause severe respiratory and systemic diseases, e.g., APEC can cause aerosacculitis, perihepatitis, polyserositis, pericarditis, egg peritonitis, salphingitis, coligranuloma, omphalitis, cellulitis, and osteomyelitis/arthritis, septicemia and other mainly extraintestinal diseases in chickens, turkeys, ducks, and other avian species; these are commonly referred as avian colibacillosis (Dziva and Stevens 2008). Multiple APEC serotypes have been associated with colibacillosis cases in the field outbreaks; however, three serotypes—O1, O2 (including O2:K2, which is one of the most common serotypes among APEC worldwide), and O78 (and especially O78:K80) account for the majority (more than 80%) of the illness (Dho-Moulin and Fairbrother 1999), (Rodriguez-Siek, Giddings et al. 2005), (Ebrahimi-Nik, Bassami et al. 2018).

Colibacillosis causes losses due to early mortality, condemnation of carcasses and reduced productivity/weight gain (Guabiraba and Schouler 2015). In this context, colibacillosis is one of the leading causes of mortality (up to 20%) and morbidity in poultry and it also results in decreased meat (2% decline in live weight, 2.7% deterioration in feed conversion ratio) and egg production (up to 20%), decreased hatching rates, and increased condemnation of carcasses (up to 43%) at slaughter—and it may be responsible for >50% mortality in young chickens. Overall, APEC costs the poultry industry hundreds of millions of dollars in economic losses worldwide (Ghunaim, Abu-Madi et al. 2014). In the United States, economic losses to the broiler industry can be as high as $40 million annually due to carcass condemnation alone. Antibiotics (tetracyclines, sulfonamides, and aminoglycosides) are frequently used to control colibacillosis in chickens. However, increasing resistance of APEC to different classes of antibiotics, including medically important antibiotics (β-lactams, colistin, and carbapenems), suggests challenges ahead in using antibiotics to control APEC infections in poultry, and highlights the need to identify novel antimicrobial strategies for managing APEC infections. Lytic E. coli bacteriophages subject of this invention may provide one such approach. Interestingly, recent studies have shown that APEC strains share some virulence genes with, and are phylogenetically related to, several E. coli serotypes that are pathogenic in humans, suggesting that APEC strains could potentially pose a health risk to humans as well (Belanger, Garenaux et al. 2011). For a review of APEC, please see (Kathayat, Lokesh et al. 2021), herein incorporated by reference in its entirety.

Pathogenic E. coli can also cause various diseases in other animal species including pets. For example, EPEC and ETEC have been strongly associated with enteric disease in young dogs (colibacillosis in dogs, usually in the lower intestines) (Beutin 1999). Pathogenic E. coli—many of the strains being multidrug resistant—have also been found in a variety of aquatic wildlife including bottlenose dolphins, harbor seals, elephant seals, and seagulls (van Elk, van Dep Bildt et al. 2007), (Simoes, Poirel et al. 2010), (Greig, Gulland et al. 2014).

### Food Safety and Environmental Contamination

As noted above, one of the main routes of infection for both humans and animals with pathogenic E. coli is though consuming foods that are contaminated with the bacteria. A variety of treatment strategies are currently employed to eliminate or significantly reduce pathogenic E. coli contamination, ranging from simple washing of foods to chemical or physical decontamination of foods. These methods vary with regard to their efficacy, cost, and impact on the flavor and aesthetic integrity of food. For example, gamma-irradiation is considered to be one of the most effective treatments, capable of reducing E. coli and various other bacteria, by 5 log 10 (Aymerich, Picouet et al. 2008). However, the process is very expensive and more effective (high) levels of gamma irradiation may adversely affect the organoleptic qualities of foods, including taste and appearance (Wheeler, Shackelford et al. 1999). Other strategies involve the application of various antibacterial chemicals, such as calcium hypochlorite, which has been reported to reduce E. coli contamination by 1.5-2.5 logs (Behrsing, Winkler et al. 2000). Unfortunately, many of those chemicals have a negative environmental impact, and their overuse may lead to a decline in efficacy (Mokgatla, Brozel et al. 1998). Importantly, in addition to targeting pathogenic bacteria, both gamma-irradiation and chemical antibacterials target beneficial bacteria, thus negatively impacting the availability of beneficial bacteria in foods (Sulakvelidze and Barrow 2005), (Abuladze, Li et al. 2008), (Carter, Parks et al. 2012), (Moye, Woolston et al. 2018).

In a somewhat different but related context, contamination of buildings, equipment, food processing facilities, medical facilities, and other facilities of strategic importance with pathogenic bacteria also remains a serious problem, which is compounded by the possibility that they may be intentionally contaminated with virulent bacteria, including pathogenic E. coli, by bioterrorists. Decontamination of such facilities also presents considerable challenges because of the increased resistance of many potentially pathogenic bacteria to traditional sanitizers, including hypochlorous acid (the active form of hypochlorite sanitizers) and benzalkonium chloride (a quaternary ammonium sanitizer) (Davidson and Harrison 2002). Also, many chemical sanitizers are corrosive and toxic and, therefore, are unacceptable for treating foods or surfaces that come into direct contact with foods. Thus, new and novel approaches are needed to aid the prevention of diseases caused by natural or intentional dissemination of pathogenic bacteria on various building materials, or by the ingestion of various foods intentionally or accidentally contaminated with pathogenic E. coli. Ideally, such approaches will be effective, safe, and economical. Lytic bacteriophages subject of this invention may provide one such approach.

In summary, and as explained above, there remains an urgent and unmet need in the art for new agents and approaches for controlling pathogenic E. coli in several critical areas, including but not limited to human and other animal clinical applications, enhancing gut resilience against pathogenic E. coli colonization and subsequent infection, food safety-related uses, environmental decontamination, and diagnostics.

### SUMMARY OF THE INVENTION

The invention meets the described needs and more by providing compositions comprising alone or in any combination novel bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249), or a combination thereof, said bacteriophages having lytic activity against pathogenic E. coli species strains. In the various embodiments, the combination may include two or more, three or more, four or more, five or more, six or more, or seven or more of the Deposited Bacteriophages. The ATCC deposit number for each phage is provided in the parenthesis. The invention additionally provides methods of using the Deposited Bacteriophages to control, reduce, or prevent colonization or contamination of processed and unprocessed food products by Targeted Bacteria, or colonization/contamination of equipment involved in the processing of the same food product(s), as well as for reducing nosocomial spread of pathogenic E. coli by decontaminating hospital centers and other healthcare facilities that may be contaminated with pathogenic E. coli. The invention additionally provides methods of using the Deposited Bacteriophages to modulate GI tract and/or enhance gut resilience against Targeted Bacteria, by reducing the incidence and/or levels of colonization in various animals (including humans) with Targeted Bacteria. The invention also provides methods of detecting the presence of Targeted Bacteria cells on processed or unprocessed food products or in other environmental or clinical samples. The invention additionally provides methods of using the Deposited Bacteriophages for the removal of antibiotic-resistant or other undesirable pathogens from medical, veterinary, animal husbandry, food processing, and other environments where they may be passed to humans or animals. The invention additionally provides for methods of using the Deposited Bacteriophages to treat, mitigate, or reduce the risk of human and/or other animal diseases caused by Targeted Bacteria.

For example, one significant need concerns the treatment of processed or unprocessed food products to reduce, eliminate or prevent colonization with undesirable bacteria such as pathogens responsible for food-borne illness and food spoilage organisms. A second critical area of need concerns the removal of undesirable bacteria from industrial environments such as food processing facilities to prevent colonization thereof. A third critical area of need concerns the removal of antibiotic resistant organisms from environments where they may be passed to humans and animals, such as hospitals, nursing homes, veterinary facilities, and other such environments. Additionally, new bacteriophage compositions and methods of using the same are needed for the treatment, mitigation, or reduction of risk of animal and human bacterial disease, particularly those diseases caused by antibiotic-resistant organisms. Furthermore, bacteriophage compositions may be used as pre-biotics or probiotics or nutritional/dietary supplements—alone or in combination with bacteria-based supplements and/or yeast-based supplements—for modulating GI microflora for various health benefits (i.e., the bacteriophages modulate GI tract microflora by specifically lysing undesirable bacteria while leaving desirable microflora intact).

The Deposited Bacteriophages are provided in order to control the growth of the Targeted Bacteria, which will reduce their ability to contaminate and colonize various environments, including but not limited to (a) raw, unprocessed food products, (b) equipment used to process or manufacture various food products, (c) various food products processed or manufactured with equipment contaminated with the Targeted Bacteria, (d) animals (including humans) contaminated/colonized with the Targeted Bacteria, (e) animal (including human) environments contaminated with the Targeted Bacteria, and (f) various processed food products for humans or animals containing ingredients contaminated with the Targeted Bacteria. The invention also provides methods for providing a prophylactic dosage(s) of the Deposited Bacteriophages, alone or in combination with antibiotics, and/or bacteria-based supplements, and/or yeast-based supplements, that may offer a subject protection against the disease caused by the Targeted Bacteria. The invention also provides methods for detecting the presence of the Targeted Bacteria in processed or unprocessed food products, and in equipment used to process or manufacture the food products. In addition, the invention provides methods of using the Deposited Bacteriophages to remove the Targeted Bacteria from medical, veterinary, animal husbandry, food processing, and other environments where they may be passed to humans or animals. Also, the invention additionally provides methods of using the bacteriophage (either as a pharmaceutical composition or nutritional supplement composition) to treat, mitigate, and reduce the risk of animal and human diseases caused by the Targeted Bacteria.

The invention meets the described needs and more by providing compositions comprising alone or in any combination novel bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249), or a combination thereof, said bacteriophages having lytic activity against the Targeted Bacteria. In the various embodiments, the combination may include two or more, three or more, four or more, five or more, six or more, or seven or more of the Deposited Bacteriophages. The invention additionally provides methods of using the Deposited Bacteriophages to control, prevent, or reduce the risk of the infection or colonization of processed and unprocessed food products by Targeted Bacteria, or colonization of equipment involved in the processing of the same food product(s). The invention additionally provides methods of using the Deposited Bacteriophages to prevent, eradicate, or reduce the levels of colonization of various animals (including humans) with Targeted Bacteria. For example, compositions comprising the Deposited Bacteriophages may be formulated as a pharmaceutical composition (e.g., drug substance) for use by animals, including humans. In another example, compositions comprising the Deposited Bacteriophages may be formulated as nutraceutical composition (e.g., dietary supplement, probiotic, or prebiotic) for use by animals, including humans. This pharmaceutical or nutraceutical composition comprising the Deposited Bacteriophage is administered (including, but not limited to, orally, and/or rectally, and/or intranasally, and/or intravenously, and/or intracerebrally, and/or ureteral, and/or intramuscularly, and/or intradermally) by an animal (including human), which lyses the Targeted Bacteria reducing colonization by the Targeted Bacteria of the animal. In another example, the same pharmaceutical or nutraceutical composition comprising the Deposited Bacteriophage is administered to an animal (including human) regularly for enhancing GI resilience against colonization with the Targeted Bacteria; if and when the Targeted Bacteria in introduced into the said animal, the composition lyses the Targeted Bacteria reducing colonization by the Targeted Bacteria of the animal and subsequent risk of infection and disease.

In yet another example, the same pharmaceutical or nutraceutical composition comprising the Deposited Bacteriophage is combined with bacteria-based and/or yeast-based preparations for enhanced ability to modulate the animal (including human) microbiome and enhance its resilience against colonization with pathogenic E. coli. The bacteria-based pharmaceutical or nutraceutical preparations can be based on, but are not limited to, strains of Lactobacillus species, including L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof. The yeast-based pharmaceutical or nutraceutical preparations can be based on, but are not limited to, strains of Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof.

The invention also provides methods of detecting the presence of Targeted Bacteria cells on processed or unprocessed food products, or equipment involved in the processing of the same food products. The invention additionally provides methods of using the Deposited Bacteriophages for the removal of antibiotic-resistant or other undesirable pathogens from medical, veterinary, animal husbandry, and other environments where they may be passed to humans or animals.

The Deposited Bacteriophage has binding specificity for Targeted Bacteria (i.e., pathogenic E. coli) and is capable of lysing Targeted Bacteria. The invention also contemplates progeny, variants, substantially equivalent bacteriophages, and bacteriophage derivative(s) of the Deposited Bacteriophages. In another embodiment, the variants of the Deposited Bacteriophage have the same phenotypic characteristics as the Deposited Bacteriophage. In another embodiment, the variants of the Deposited Bacteriophage have the same lytic specificity for pathogenic E. coli as the Deposited Bacteriophage. In a still another embodiment, the variants of the Deposited Bacteriophage differ genetically from the Deposited Bacteriophage by a single genetic event including but not limited to silent mutations, inversions, deletions, insertions, polymorphisms, or point mutations but still retain the same phenotypic characteristics and lytic specificity for pathogenic E. coli as the Deposited Bacteriophage. In many embodiments, the progeny may be variants of the Deposited Bacteriophage. In one embodiment, the invention provides progeny of the Deposited Bacteriophage having minor variation(s) in the genomic sequence and polypeptides encoded thereby while retaining the same general genotypic and/or phenotypic characteristics as the Deposited Bacteriophage. In particular, these progenies are the result of successive passaging of the Deposited Bacteriophage where the variants accumulate silent mutations, conservative mutations, minor deletions, and/or minor replications of genetic material. The progeny described herein of the Deposited Bacteriophage retain the phenotypic characteristics of the Deposited Bacteriophage, in a preferred embodiment, the progeny retains lytic activity against the Target Bacteria.

In an embodiment, the invention provides derivatives of the Deposited Bacteriophage comprising substances that constitute subunits or expression products of the Deposited bacteriophage or its progeny, including (but not limited to) phage nucleic acids, partial or complete phage genes, gene expression products (e.g., exopolysaccharide degrading enzymes), and structural components (e.g., polyribonucleotide(s) and polydeoxyribonucleotide(s), including modified or unmodified bacteriophage DNA, cDNA, mRNA and synthetic polynucleotide sequences, as well as DNA/RNA hybrids.) In another embodiment, the invention provides modified polynucleotides (e.g., phosphorylated DNAs) of the Deposited Bacteriophages.

In an embodiment, the invention provides the use of the Deposited Bacteriophage, and its progeny and derivatives, to control the growth on, or colonization of, processed and unprocessed food products by Targeted Bacteria, or the colonization of buildings and equipment, particularly those associated with the processing of the same food product. The invention also provides methods of identifying Targeted Bacteria as a bacterial diagnostic and/or detecting the presence of Targeted Bacteria on processed or unprocessed food products, or equipment or buildings such as those involved in the processing of the same food products. The invention further provides methods of using the Deposited Bacteriophages and their progeny and derivatives for the removal of antibiotic-resistant or other undesirable pathogens from medical, veterinary, animal husbandry, or any additional environments where they may be passed to humans or animals. The invention additionally provides for methods of using the Deposited Bacteriophages and their progeny and derivatives to treat, mitigate, and/or reduce the risk of human and animal diseases caused by Targeted Bacteria. The Deposited Bacteriophages and their progeny and derivatives are administered for the methods of the invention as a homogenous phage administration, or alternatively as a component of a multi-phage composition comprising several bacteriophages and/or other bacteria-based or yeast-based compositions. These methods of use are provided with greater particularity infra.

In any one embodiment, one possessing the Deposited Bacteriophage will inevitably be in possession of progeny of the Deposited Bacteriophages. Furthermore, after successive sub culturing (e.g., over 10 passages) of the Deposited Bacteriophages, progeny having genetic variations within the scope of “closely related” organisms as descried by Tenover et al. (Tenover, Arbeit et al. 1995), or “Same Species” as described by (Olm 2017), (Jain, Rodriguez et al. 2018), are present.

In one embodiment, the invention comprises bacteriophages substantially equivalent to the Deposited Bacteriophages—bacteriophages that are “indistinguishable” from or “closely related” to the Deposited Bacteriophages as these terms are defined in Tenover et al. (Tenover, Arbeit et al. 1995), or fall under the “Same Species” designation as described by (Olm 2017) and (Jain, Rodriguez et al. 2018). In any of the foregoing embodiments, the composition comprises at least one, two, three, four, five, six, seven, or all eight of the Deposited Bacteriophages.

In another embodiment, a nutraceutical composition may comprise at least one of the Deposited Bacteriophages. The nutraceutical composition may further comprise an excipient, carrier, stabilizer, flavoring, or colorant agent.

The present invention is directed to novel phage compositions useful in treating food products to minimize or eliminate bacterial contamination by pathogenic E. coli bacteria. The phage compositions can be formulated with suitable carriers.

The compositions of the present invention may be used for human, veterinary, agricultural or aquacultural purposes. Furthermore, the compositions as described herein may be used for environmental applications. The composition may be used within a cream, lotion, or gel, be admixed with a pharmaceutical carrier and administered topically, orally, rectally, nasally, used as an inhalant, or the antibacterial composition may be added to a feed for animal, aquatic, or avian uses.

In another embodiment of the invention, isolated progeny of the deposited bacteriophage derived from the deposited bacteriophage.

Another embodiment of the invention comprises isolated progeny of the progeny of the deposited bacteriophage.

One embodiment of the invention comprises at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249), deposited to the American Type Culture Collection, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains.

Another embodiment of the invention comprises at least one progeny of bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249), deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains.

Still another embodiment comprises at least one derivative of the bacteriophage of isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains, said derivative comprising nucleic acids, partial or complete genes, gene expression products, structural components, or one or more combinations thereof.

In any of the foregoing embodiments, the composition may comprise at least one derivative of the progeny bacteriophage of isolated bacteriophage ECML-1 (PTA-127245), ECML-10 (PTA-127246), ECML-123-2 (PTA-121408), ECML-183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains, said derivative comprising nucleic acids, partial or complete genes, gene expression products, structural components, or one or more combinations thereof.

In any of the foregoing embodiments, a composition may comprise an isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, progeny, derivatives, and mixtures thereof. In some embodiments, the composition may be a pharmaceutical composition, nutraceutical product, dietary supplement, probiotic, and/or prebiotic.

In some embodiments, the composition may be a concentrated aqueous solution or dried powder preparation. In any of the embodiments, the composition comprises one or more of the following ingredients: deionized water, buffer solution, preferably Tris-HCl pH 7.0-7.5, mineral water, sucrose, glycerol, trehalose, dextran, polyethylene glycol, sorbitol, cellulose, tapioca dextrin, hydroxypropyl methylcellulose, gellan gum, gelatin, casein, NaCl, MgSO4, or a mixture thereof.

One embodiment comprises a method for the treatment, mitigation, and/or reduction of risk of food borne illnesses caused by pathogenic E. coli strains, comprising contacting a food product or products with a microbial growth inhibiting effective amount of a bacteriophage composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains.

One embodiment comprising a method for the reduction of the incidence of food borne illnesses caused by pathogenic E. coli strains, comprising contacting a food product or products with a microbial growth inhibiting effective amount of a bacteriophage composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains.

In several embodiments, the contacting described in the methods herein comprises (i) spraying or misting or fogging the bacteriophage composition on the food product(s), or (ii) dipping or soaking the food product(s), or (iii) adding, injecting, or inserting the bacteriophage composition into the food product(s) or food product packaging, with the solution containing a concentration of the bacteriophage composition sufficiently high to inhibit the growth of pathogenic E. coli strains.

In any embodiment, a method for reducing the risk of bacterial infection (e.g., gastrointestinal disease, urinary tract infection, etc.) or sepsis in a person colonized with bacteria comprising treating the colonized person with a pharmaceutical composition containing bacteriophage of one or more strains of the Deposited Bacteriophage which produce lytic infections in said bacteria, wherein said treatment occurs prior to said colonized person developing an illness due to said bacteria a said treatment reduces the risk of bacterial infection or sepsis in said colonized person, and wherein said treatment of the colonized person reduces the level of colonization with bacteria susceptible to the bacteriophage by at least 50%, wherein said composition is administered intravesicularly, topically, orally, rectally, intraurethrally, ocularly, optically, vaginally, topically, nasally, or via inhalation. Additionally, said bacteria is pathogenic E. coli. In a more preferred embodiment, the bacteriophage composition is an oral tablet, capsule, enteric coated gel cap, tablet, gummy, liquid or syrup, a nasal aerosol, a throat wash, a mouth wash or gargle, a toothpaste, a topical ointment, or rinse solution. In another embodiment, the colonized person is a person having a diarrhea, and the bacteriophage produce lytic infections in bacteria capable of causing diarrhea. In yet another embodiment, the colonized person is a person having urinary tract infection, and the bacteriophage produce lytic infections in bacteria capable of causing UTI.

In any embodiment, a method for reducing the risk of bacterial infection or sepsis in a person not colonized with pathogenic E. coli bacteria comprising treating the person with a pharmaceutical composition containing bacteriophage of one or more strains of the Deposited Bacteriophage which produce lytic infections in said pathogenic E. coli bacteria, wherein said treatment occurs prior colonization of the person or development an illness due to said bacteria and said treatment reduces the risk of bacterial infection or sepsis in person, and wherein said treatment of the person prevents the colonization with bacteria susceptible to the bacteriophage, wherein said composition is administered intravesicularly, intraurethrally, vaginally, topically, orally, rectally, ocularly, optically, nasally, or via inhalation. In a more preferred embodiment, the bacteriophage composition is an oral tablet, capsule, enteric-coated gel cap, enteric-coated tablet, syrup, gummy, liquid, a nasal aerosol, a throat wash, a mouth wash or gargle, a toothpaste, rinse solution, and a topical ointment. In another embodiment, the person is a person having a diarrhea and bloating, and the bacteriophage produce lytic infections in pathogenic E. coli bacteria causing these symptoms. In yet another embodiment, the person is a person having UTI, and the bacteriophage produce lytic infections in pathogenic E. coli bacteria causing these symptoms.

In another embodiment of the invention, a composition may comprise at least one of the bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains.

In another embodiment, the composition further comprises a pharmaceutically acceptable carrier wherein the pharmaceutically acceptable carrier is an aerosol, a paste, a powder, a syrup, rinse solution, or an injectable formulation.

Another embodiment comprises the use of a bacteriophage composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains for reducing the risk of food borne illnesses caused by pathogenic E. coli strains comprising contacting a food product or products with a microbial growth inhibiting effective amount of said bacteriophage composition. In a preferred embodiment, said contacting comprises spraying or misting or fogging the bacteriophage composition on poultry meat (before or after grinding), wheat kernels, flour, or other food product(s), or by dipping or soaking these product(s) in a solution containing a concentration of the bacteriophage composition sufficiently high to inhibit the growth of pathogenic E. coli strains, or adding, injecting or inserting the bacteriophage composition in said concentrations into the food product(s) or package which contains said food product(s).

In any embodiment, the pharmaceutical composition is formulated as a capsule, tablet, chewable composition, syrup, rinse solution, or gel. In any embodiment, the capsule may be an enteric-coated gel capsule.

In another embodiment, this invention provides a method of treating, mitigating, and/or reducing the risk of diseases caused by pathogenic E. coli by administering an effective amount of a composition according to the invention to a person in need thereof. In one or more modes of this embodiment, the composition per the invention may be a dietary supplement, a feed additive, a nutraceutical composition, or a pharmaceutical (drug) composition. Such compositions may comprise one or more isolated bacteriophages having lytic activity against pathogenic E. coli strains. The bacteriophages may be selected from a group consisting of ECML-1 deposited under ATCC Deposit Accession No. PTA-127245, ECML-10 deposited under ATCC Deposit Accession No. PTA-127246, ECML-123-2 deposited under ATCC Deposit Accession No. PTA-121408, ECML-183-2 deposited under ATCC Deposit Accession No. PTA-127247, ECML-359 deposited under ATCC Deposit Accession No. PTA-121406, ECML-363 deposited under ATCC Deposit Accession No. PTA-121407, ECML-606-1 deposited under ATCC Deposit Accession No. PTA-127248, ECCR-664-1 deposited under ATCC Deposit Accession No. PTA-127249, or a combination thereof. In any of these embodiments, the person may be an adult, infant, or child, and the child may be less than 5 years of age. In any of these embodiments, the composition may comprise the one or more bacteriophage in amounts of 10⁶ and 10¹² PFU. The composition used in these embodiments may comprise one or more of isolated progeny of the aforementioned bacteriophages. Such progeny may have average nucleotide identity over genome of at least 80%, at least 90%, at least 91%, at least 92%, at least 93%, at least 94%, at least 95%, at least 96%, at least 97%, at least 98%, or at least 99% relative to the aforementioned bacteriophages. Further, the progeny will have lytic activity against pathogenic E. coli strains. In these embodiments, the composition may be formulated as a capsule, gel capsule, enteric coated capsule, microgel, suppository, rinse solution, tablet, chewable composition, syrup, or gel.

In variants of these embodiments, the composition may further comprise a probiotic bacterium, a probiotic yeast, or both. Such probiotic bacteria may be Lactobacillus species, preferably selected from the group consisting of L. acidophilus, L. rhamnosus, L. gasseri, L. reuteri, L. bulgaricus, L. plantarum, L. johnsonii, L. paracasei, L. casei, L. salivarius, L. lactis, or the probiotic bacteria may be Bifidobacterium species, preferably selected from the group consisting of B. bifidum, B. longum, B. breve, B. infantis, B. lactis, B. adolescentis, or the probiotic bacteria may be selected from the group consisting of Streptococcus thermophilus, Bacillus cerus, Bacillus subtilis, Enterococcus faecalis, Enterococcus faecium, or a combination one or more of these bacterial species. The composition used in this embodiment may comprise the probiotic bacteria in an amount of 100-10 billion Colony Forming Units (CFU). Such probiotic yeast may be selected from the group consisting of Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof. The compositions used in this embodiment may comprise probiotic yeast in an amount of 100-10 billion Colony Forming Units (CFU).

In variants of these embodiments, the composition may further comprise one or more of other ingredients that enhance prophylactic or therapeutic effect of the deposited bacteriophages. In exemplary embodiments, the pharmaceutical and/or nutraceutical composition is combined with proanthocyanidin, and/or cranberry juice extract, and/or D-mannose, and/or vitamins (preferably vitamin C), and/or acetic acid, and/or celery extract to treat, mitigate, or reduce the risk of urinary tract infections caused by pathogenic E. coli (UPEC in particular). Typically, the composition is administered orally, intravesicularly, intraurethrally, vaginally, topically, rectally, ocularly, optically, nasally, or via inhalation.

In at least one embodiment, the invention provides a method for the reduction in the incidence of food borne illnesses caused by pathogenic E. coli strains comprising contacting food processing equipment with a microbial growth inhibiting effective amount of a bacteriophage composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) (deposited to the ATCC), these bacteriophage having lytic activity against pathogenic E. coli strains, and variants thereof, wherein the variants retaining the phenotypic characteristics of the bacteriophage, where the bacteriophage and variants thereof have lytic activity against pathogenic E. coli strains. In any such embodiment, the contact may comprise spraying or misting or fogging the bacteriophage composition on the food processing equipment, dipping or soaking the food processing equipment in a solution containing a concentration of the bacteriophage composition sufficiently high to inhibit the growth of pathogenic E. coli strains, or adding, injecting or inserting the bacteriophage composition into the food processing equipment; or spraying or misting the bacteriophage composition on a surface used in food processing. In exemplary embodiments, the foods are wheat kernels (including outer shell, bran, germ, and endosperm), flour, poultry meat, red meat, shellfish, fruits and vegetables, dairy products, healthy drinks, and ready-to-eat foods.

In the embodiments of the invention described herein, the pathogenic E. coli strain may be STEC, EHEC, EPEC, ETEC, EAEC, EIEC, DAEC, UPEC, MNEC, or a combination thereof. In particular embodiments, the pathogenic E. coli strain is avian pathogenic E. coli, sepsis-associated E. coli, mammary pathogenic E. coli, endometrial pathogenic E. coli, and necrotoxigenic E. coli, or a combination thereof. In exemplary embodiments, the pathogenic E. coli strains are STEC, EHEC, EPEC, ETEC, EAEC, EIEC, DAEC, UPEC, MNEC, avian pathogenic E. coli, sepsis-associated E. coli, mammary pathogenic E. coli, endometrial pathogenic E. coli, necrotoxigenic E. coli, or a combination thereof.

In numerous embodiments, the invention provides methods for reducing colonization by pathogenic E. coli bacteria strains of a subject by administration of an effective amount of a nutraceutical composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) (deposited to the ATCC), the bacteriophage having lytic activity against pathogenic E. coli strains, and variants thereof, where the variants retain the phenotypic characteristics of the bacteriophage, where the bacteriophage and variants thereof have lytic activity against pathogenic E. coli strains. In typical embodiments, the nutraceutical composition is formulated as a capsule, tablet, chewable composition, syrup, or gel. In particular embodiments, the capsule is a gel capsule.

In many of these embodiments, the subject may be an adult, infant, or child, and the child may be less than 5 years of age.

In several embodiments, a method for modulating an animal (including human) microbiome by reducing colonization by pathogenic E. coli bacteria strains may comprise administration of an effective amount of a composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) (deposited to the ATCC), the bacteriophage having lytic activity against pathogenic E. coli strains, and variants thereof, where the variants retain the phenotypic characteristics of said bacteriophage and where the bacteriophage and variants thereof have lytic activity against pathogenic E. coli strains. In particular embodiments, the method reduces colonization of the gastrointestinal tract, vagina, skin, or a combination thereof.

In several embodiments, a method for maintaining healthy gut microflora by modulating an animal (including human) microbiome by reducing colonization by pathogenic E. coli bacteria strains may comprise administration of an effective amount of a composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains. In one embodiment, the method reduces colonization of the gastrointestinal tract, urinary tract, vagina, skin, or a combination thereof.

In several embodiments, a dietary supplement, feed additive, nutraceutical, and/or pharmaceutical (drug) composition comprises one or more of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains. In some aspects, the dietary supplement, feed additive, nutraceutical, and/or pharmaceutical (drug) composition includes at least one probiotic bacteria and/or at least one probiotic yeast. The at least one probiotic bacteria comprises preferably Lactobacillus species, preferably L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof. The at least one probiotic yeast comprises preferably Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof.

In any of the foregoing embodiments, the composition is a pharmaceutical composition, nutraceutical composition, dietary supplement, probiotic, and/or prebiotic.

In any of the foregoing embodiments, the animal may be already colonized by a pathogenic E. coli strain or strains.

In any of the foregoing embodiments, the animal may not be colonized by a pathogenic E. coli strain or strains.

In any of the foregoing embodiments, the bacteriophage may be present in a composition in an amount of 10⁶ and 10¹¹ PFU. In any of the foregoing embodiments, the animal may be a human.

In any of the foregoing embodiments, the human may be an adult, infant, or child, and the child may be less than 5 years of age.

In any of the foregoing embodiments, the combination may include two or more, three or more, four or more, five or more, six or more, or seven or more of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC and variants thereof.

### BRIEF DESCRIPTION OF THE FIGURES

### Restriction Fragment Length Polymorphism (RFLP) Analysis of Deposited Bacteriophages

FIG. 1 shows RFLP patterns of the eight Deposited Bacteriophages resulting from digestion with EcoRV.

FIG. 2 shows a Force Graph activity visualization of the Deposited Phages against 738 E. coli strains.

### DETAILED DESCRIPTION OF THE PREFERRED EMBODIMENTS

### Definitions

The terminology used in the description of the invention herein is for the purpose of describing particular embodiment only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

“Administration,” as used herein, refers broadly to any means by which a composition is given to a subject, be it a patient, healthy or diseased human, or other healthy or diseased animal species.

“ATCC,” as used herein, refers to the American Type Culture Collection, located in Manassas, Virginia, USA, and Gaithersburg, Maryland, USA.

“Bacteriophage composition,” as used herein refers broadly to a composition comprising, or alternatively consisting essentially of, or alternatively consisting of, the Deposited Bacteriophage. A “bacteriophage composition” as used herein does not include the Deposited Bacteriophage as it exists in its natural environment prior to isolation and/or substantial purification. Further, a composition may comprise, consist of, or essentially consist of at least one of the Deposited Bacteriophages. Alternatively, the compositions as described herein may comprise, consist of, or essentially consist of at least one, two, three, four, five, six, seven, or all eight of the Deposited Bacteriophages.

“Bacteriophages substantially equivalent to the Deposited Bacteriophages,” as used herein, refers broadly to those bacteriophages that are “indistinguishable” from or “closely related” to the Deposited Bacteriophages as these terms are defined in (Tenover, Arbeit et al. 1995) for PFGE patterns, and/or in (Olm 2017) (Jain, Rodriguez et al. 2018) for full genome sequence analyses. For example, Tenover et al. describes that organisms are “genetically indistinguishable if their restriction patterns have the same numbers of bands and the corresponding bands are the same apparent size.” (Tenover at page 2235). Epidemiologically, these organisms are “all considered to represent the same strain, i.e., isolates demonstrating the common outbreak pattern represent the outbreak strain.” (Tenover at page 2235). Accordingly, under Tenover, a particular organism is “indistinguishable” from itself or its clone. Tenover describes that an organism is “closely related” if its “PFGE pattern differs from the outbreak pattern by changes consistent with a single genetic event, i.e., a point mutation or an insertion or deletion of DNA. Such changes typically result in two to three band differences.” (Tenover at page 2235). Tenover states that such two to three band differences “have been observed in strains of some species when they are cultured repeatedly over time or isolated multiple times from the same patient.” (Tenover at page 2235). Accordingly, under Tenover et al., progeny of an organism (e.g., descendants of the organism created by serial passage of the organism), for example, are “closely related” to the parent organism (Tenover, Arbeit et al. 1995). For genome-based sequence analysis, Olm and Jain et al. (Olm 2017), (Jain, Rodriguez et al. 2018) define as “Same Species” organisms with the average nucleotide identity over genome (gANI) of ANI≥95%, said criteria used herein for defining bacteriophages substantially equivalent to the Deposited Bacteriophages.

“Colonization” or “colonized,” as used herein, refers broadly to the presence of Targeted Bacteria on foodstuff(s), or environmental surface(s), or in vivo such as in the gastrointestinal tract, urinary tract, or skin of a mammalian organism without perceptible significant alteration other than the presence of bacteria. The presence of the Targeted Bacteria is non-transient, e.g., the Targeted Bacteria are persistent in or on the colonized foodstuff(s) or environmental surface(s), or growing or multiplying in vivo. The terms “colonization” and “colonized” stand in contrast to the terms “infection” or “infected” which are commonly understood to require perceptible deleterious alteration as part of their definition. “Colonization” and “colonized” may also refer to the presence of bacteria in or on a human or animal without perceptible damage, alteration, or disease.

“Deposited Bacteriophage,” as used herein, refers broadly to isolated bacteriophages ECML-1 deposited with the ATCC on Jan. 14, 2022 (Deposit Accession No. PTA-127245), ECML-10 deposited with the ATCC on Jan. 14, 2022 (Deposit Accession No. PTA-127246), ECML-123-2 deposited with the ATCC on Jul. 25, 2014 (Deposit Accession No. PTA-121408), ECML183-2 deposited with the ATCC on Jan. 14, 2022 (Deposit Accession No. PTA-127247), ECML-359 deposited with the ATCC on Jul. 25, 2014 (Deposit Accession No. PTA-121406), ECML-363 deposited with the ATCC on Jul. 25, 2014 (Deposit Accession No. PTA-121407), ECML-606-1 deposited with the ATCC on Jan. 14, 2022 (Deposit Accession No. PTA-127248), ECCR-664-1 deposited with the ATCC on Jan. 14, 2022 (Deposit Accession No. PTA-127249).

Bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) were deposited with the American Type Culture Collection (ATCC) under the Budapest Treaty on the International Recognition of the Deposit of Microorganisms for the Purposes of Patent Procedure.

Additionally, “Deposited Bacteriophage,” as used herein, refers broadly to isolated bacteriophages ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited with the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains. In the various embodiments, the Deposited Bacteriophage may be used in combinations including two or more, three or more, four or more, five or more, six or more, or seven or more of the Deposited Bacteriophages.

All of the Deposited Bacteriophages described herein are lytic not lysogenic phages. The Deposited Bacteriophages have lytic activity against pathogenic E. coli strains.

“Derivatives,” as used herein, refers broadly to all substances that constitute subunits or expression products of the Deposited Bacteriophage or its progeny, including (but not limited to) phage nucleic acids, partial or complete phage genes, gene expression products, and structural components. For example, derivatives of the invention mean polyribonucleotide(s) and polydeoxyribonucleotide(s), including modified or unmodified bacteriophage DNA, cDNA, mRNA and synthetic polynucleotide sequences, as well as DNA/RNA hybrids. In another example, polynucleotides of the invention encompass modified polynucleotides, such as for example phosphorylated DNAs. In yet another example, gene expression products mean phage-encoded exopolysaccharide degrading enzymes, holins, and lysins.

“Effective amount,” as used herein, refers broadly to the amount of an isolated bacteriophage that, when administered to an animal (including, but not limited to swine, cattle, poultry, and sheep) and/or human patient for treating a disease, is sufficient to affect such treatment for the disease. The effective amount can be an amount effective for treatment, mitigation, and/or reduction of risk. The effective amount can be an amount effective to reduce the incidence of illnesses, an amount effective to reduce incidence of illnesses, to reduce the severity of illness, to eliminate infection, to slow the development of the infection, to reduce the risk of development of infection or colonization. The “effective amount” can vary depending on the disease and its severity and the age, weight, medical history, predisposition to conditions, preexisting conditions, of the animal or human patient to be treated. The term “effective amount” is taken to be synonymous with “therapeutically effective amount” for purposes of this invention.

“Isolated,” as used herein, refers broadly to material removed from its original environment in which it naturally occurs, and thus is altered by the hand of man from its natural environment. Isolated material may be, for example, foreign nucleic acid included in a vector system, foreign nucleic acid contained within a host cell, or any material which has been removed from its original environment and thus altered by the hand of man. Isolated material further encompasses bacteriophage specific for the Targeted Bacteria or particular Targeted Bacteria isolates, isolated and cultured separately from the environment in which it was located, where these isolates are present in purified compositions that do not contain any significant amount of other bacteriophage or bacterial strains, respectively.

“Mammal” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, chimpanzees, chinchillas, cattle, dogs, goats, gorillas, horses, humans, lemurs, llamas, mice, non-human primates, pigs, rats, sheep, shrews, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington DC, which is hereby incorporated by reference.

“Avian” as used herein, refers broadly to any and all warm-blooded avian animals including poultry (chickens, ducks, turkeys, geese, guinea fowl and squabs), wildlife, waterfowl, psittacine birds, and game birds.

“ORF,” as used herein, refers broadly to an Open Reading Frame which is an in-frame sequence of codons that (in view of the genetic code) correspond to or encode a protein or peptide sequence. Two ORFs correspond to each other if the sequences or their complementary sequences encode the same amino acid sequences. An ORF sequence, operably associated with appropriate regulatory sequences, may be transcribed and translated into a polypeptide in vivo. A polyadenylation signal and transcription termination sequence will usually be located 3′ to the coding sequence.

“Patient” as used herein, refers broadly to any animal, including humans, who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient can be a clinical patient such as a human or a veterinary patient such as a companion, domesticated, livestock, exotic, or zoo animal. Animals can be mammals, reptiles, birds, amphibians, or invertebrates.

“Progeny,” as used herein, refers broadly to replicates of the Deposited bacteriophage, including descendants of the Deposited bacteriophage created by serial passage of the Deposited bacteriophage or by other means well known in the art, or bacteriophage whose RFLP profiles are substantially equivalent to the RFLP profile of the Deposited bacteriophage or those with ANI≥95%. The term substantially equivalent is used to describe variability between organisms in accordance with the standards advanced by Tenover et al. for RFLP patterns (Tenover, Arbeit et al. 1995) or by Olm and Jain et al. for genome sequence analysis (Olm 2017), (Jain, Rodriguez et al. 2018). For example, Tenover teaches the acceptable levels of variation that may be seen when the genomes of identical propagated organisms are electrophoretically analyzed following restriction enzyme digestion. Bacteriophages “substantially equivalent” to the Deposited Bacteriophages are “indistinguishable” from or “closely related” to the Deposited Bacteriophages. Tenover et al. describe a system for interpreting chromosomal DNA Restriction Enzyme digest patterns (“RFLP”) using Pulsed-Field Gel Electrophoresis (PFGE). (Tenover at page 2233). In particular, Tenover et al. set forth various categories of genetic and epidemiologic relatedness including those organisms that are “indistinguishable” from or “closely related” to each other. While Tenover et al. provide a schematic (prophetic) example of PFGE patterns of genetically related bacteria, the same principles being applied for bacteria also apply to bacteriophage, because Tenover is analyzing genomic DNA. For genome sequence-based analysis, Olm (Olm 2017) and Jain et al. (Jain, Rodriguez et al. 2018) define as “Same Species” organisms with ANI≥95% said criteria used herein for defining “Progeny” of the Deposited Bacteriophages.

“Recombinant bacteriophage,” as used herein, refers broadly to all genetically modified versions of the Deposited Bacteriophage or its progeny, obtained by serial passaging (in vivo or in vitro) or genetic manipulations of the Deposited Bacteriophage or its progeny. Such manipulations include, but are not limited to, serial passaging for selection of desirable properties, and/or introducing genes or gene cassettes encoding alternative proteins or nonfunctional proteins, or noncoding nucleotide sequences into the genome of the Deposited Bacteriophages or removing certain genes or gene cassettes from the Deposited Bacteriophages.

“Substantially pure,” as used herein refers broadly to material essentially free of any similar macromolecules that would normally be found with it in nature. For example, a substantially pure bacteriophage is in a composition that contains no more than 1% of other bacteriophages.

“Targeted Bacteria,” as used herein, refers broadly to the pathogenic E. coli strains including STEC, EHEC, EPEC, ETEC, EAEC, EIEC, AIEC, DAEC, UPEC, MNEC, avian pathogenic E. coli, sepsis-associated E. coli, mammary pathogenic E. coli, endometrial pathogenic E. coli, necrotoxigenic E. coli, or a combination thereof.

“Pathogenic E. coli,” as used herein, refers broadly to the pathogenic E. coli strains including STEC, EHEC, EPEC, ETEC, EAEC, EIEC, AIEC, DAEC, UPEC, MNEC, avian pathogenic E. coli, sepsis-associated E. coli, mammary pathogenic E. coli, endometrial pathogenic E. coli, necrotoxigenic E. coli, or a combination thereof.

“Therapy” or “therapeutic,” as used herein, refers broadly to treating a disease, arresting, or reducing the development of the disease or its clinical symptoms, and/or relieving the disease, causing regression of the disease or its clinical symptoms. Therapy encompasses prophylaxis, prevention, treatment, cure, regimen, remedy, minimization, reduction, alleviation, reduction of risk, mitigation, and/or providing relief from a disease, signs, and/or symptoms of a disease. Therapy encompasses alleviating a disease state or preventing the occurrence or reoccurrence of a disease state. Therapy encompasses an alleviation of signs and/or symptoms in patients with ongoing disease signs and/or symptoms, e.g., of infection. Therapy also encompasses “prophylaxis” and “prevention”. Prophylaxis includes preventing or reducing the risk of disease occurring subsequent to treatment of a disease in a patient or reducing the incidence or severity of the disease in a patient. The term “reduced”, for purpose of therapy, refers broadly to the clinically significant reduction in signs and/or symptoms. Therapy includes treating relapses or recurrent signs and/or symptoms, e.g., of colonization. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease.

“Variants,” as used herein, refers broadly to bacteriophages that share the same phenotypic characteristics of the Deposited Bacteriophage and share the same lytic activity of the Deposited Bacteriophages against the Targeted Bacteria. Variants also include bacteriophages that are “substantially equivalent” to the Deposited Bacteriophages or are “indistinguishable” from or “closely related” to the Deposited Bacteriophages as described in Tenover et al. for RFLP-based analysis (Tenover, Arbeit et al. 1995), or fall under the “Same Species” classification (i.e., ANI≥95%) as defined by Olm (Olm 2017) and Jain et al. (Jain, Rodriguez et al. 2018) for sequence-based analysis. In some aspects, the variants have an average nucleotide identity over genome of ≥80%, ≥90%, or ≥95% relative to the Deposited Bacteriophage. In some aspects, the variants have an average nucleotide identity over genome of 90%, 93%, 95%, 97%, 98%, or 99% relative to the Deposited Bacteriophage.

“Microgel,” as used herein, refers to cross-linked or interwoven three-dimensional polymeric networks. The microgels may be hydrogels, which may absorb and retain large amounts of water. The Deposited Bacteriophage or derivatives thereof may be encapsulated in, embedded in, cross-linked to, etc. the microgel. The microgels may undergo abrupt volume changes in response to environmental factors such as temperature, ionic strength, and pH. Accordingly, the physical and chemical properties of the microgel may be customized for controlled release of encapsulated amounts of the Deposited Bacteriophage or derivatives thereof in particular environmental conditions. The microgel may be a biodegradable microgel that includes degradable linkages in the polymer or cross-linker.

### Genome Analysis and Average Nucleotide Identity of the Deposited Bacteriophages

All deposited bacteriophages were sequenced and assembled. Seven bacteriophages were sequenced on MiSeq with a read length of 2×250 bp. Reads were trimmed for Illumina adapter, length (≥50 bp), quality (q≥20), and assembled using Spades v3.12.0 or Unicycler v0.4.8.0r. Bacteriophage ECCR-664-1 was sequenced on MinION using Native genome sequencing workflow and assembled using Canu (Koren, Walenz et al. 2017) and polished with Oxford Nanopore Technologies' Medaka tool. For seven samples, ECML-1, ECML-10, ECML-123-2, ECML-183-2, ECML-359, ECML-363, and ECML-606-1, the reads assembled into a single circular contig indicating a complete genome assembly was achieved. The assembly was further polished with pilon (Walker, Abeel et al. 2014) to obtain the final genome sequence. Assembly and polishing of ECCR-664-1 reads returned 3-contigs of total length 169532 nt. The assembled genome sizes are presented in Table 1.

TABLE 1
Genome size of the Deposited Bacteriophages

| Phage | Genome length (bp) |
|---|---|
| ECML-1 | 88,695 |
| ECML-10 | 138,298 |
| ECML-123-2 | 163,097 |
| ECML-183-2 | 149,818 |
| ECML-359 | 169,468 |
| ECML-363 | 167,029 |
| ECML-606-1 | 94,968 |
| ECCR-664-1 | 134,406 |

The GenBank accession numbers for the genome sequences of the deposited bacteriophages are summarized in Table 2.

TABLE 2
ATCC deposit and GenBank accession numbers
of the deposited bacteriophages

| | ATCC | GenBank | GenBank | |
|---|---|---|---|---|
| | Deposit | Accession | Deposit | Released to |
| Phage ID | Number | Number | Date | Public Date |
| ECML-1 | PTA-127245 | OL631478 | Jan. 14, 2022 | Jan. 2, 2024 |
| ECML-10 | PTA-127246 | OL631479 | Jan. 14, 2022 | Jan. 2, 2024 |
| ECML-123-2 | PTA-121408 | OL631480 | Jul. 25, 2014 | Jan. 2, 2024 |
| ECML-183-2 | PTA-127247 | OL631481 | Jan. 14, 2022 | Jan. 2, 2024 |
| ECML-359 | PTA-121406 | OL631482 | Jul. 25, 2014 | Jan. 2, 2024 |
| ECML-363 | PTA-121407 | OL631483 | Jul. 25, 2014 | Jan. 2, 2024 |
| ECML-606-1 | PTA-127248 | OL631484 | Jan. 14, 2022 | Jan. 2, 2024 |
| ECCR-664-1 | PTA-127249 | OL631257 | Jan. 14, 2022 | Jan. 2, 2024 |

The SEQ ID NO.s for the genome sequences of the deposited phages are summarized in Table 3.

TABLE 3
SEQ ID NO.s of the deposited bacteriophages

| SEQ ID NO. | Phage ID |
|---|---|
| 1 | ECML-363 complete genome |
| 2 | ECCR-664 contig-1 |
| 3 | ECCR-664 contig-2 |
| 4 | ECCR-664 contig-3 |
| 5 | ECML-1 complete genome |
| 6 | ECML-123-2 complete genome |
| 7 | ECML-606-1 complete genome |
| 8 | ECML-183-2 complete genome |
| 9 | ECML-10 complete genome |
| 10 | ECML-359 complete genome |

The assemblies were compared to the NCBI RefSeq Genome database to identify the closest neighbor. The strain delineation was assessed by calculating average nucleotide identity over genome (gANI) using OrthoANIu algorithm (Yoon, Ha et al. 2017). The assembled contigs were analyzed in a pairwise fashion using a locally installed OrthoANIu on an in-house Linux server. The OrthoANIu tool was run with the default setting to report ≥80% gANI for each genome pair. Sequence delineation was interpreted based on previously published cutoff values presented by Olm et. al. (Olm, Brown et al. 2017) and Jain et al. (Jain, Rodriguez et al. 2018). gANI<80% was considered divergent whereas gANI>99.9 was used to determine if the two sequences were identical as presented below (Olm, Brown et al. 2017), (Jain, Rodriguez et al. 2018).

| | |
|---|---|
| ANI <80% | Divergent genomes |
| ANI 80%-90% | Moderately divergent |
| ANI 90%-95% | Closely related taxa |
| ANI ≥95% | Same species |
| ANI ≥98% | Same genetic clade |
| ANI ≥99.9% | Same strain |

Based on the classification by Olm et. al. (Olm, Brown et al. 2017) and Jain et. al. (Jain, Rodriguez et al. 2018) bacteriophages with ANI≥95% could be considered “same species” and bacteriophages with a gANI<80% could be considered “divergent.” When applying this classification to the deposited bacteriophages, all deposited bacteriophages are distinct from each other (Table 4). All the bacteriophages are divergent from each other except for the pair ECML-123-2 and ECML-363, which has a gANI value of 95% indicating these are the members of the same species.

TABLE 4
Average nucleotide identity (gANI) of the deposited bacteriophages (%)

| | ECML- | ECML- | ECML- | ECML- | ECML- | ECCML- | ECML- | ECCR- |
|---|---|---|---|---|---|---|---|---|
| | 1 | 10 | 123-2 | 183-2 | 359 | 363 | 606-1 | 664-1 |
| ECML-1 | 100% | <80% | <80% | <80% | <80% | <80% | <80% | <80% |
| ECML-10 | | 100% | <80% | <80% | <80% | <80% | <80% | <80% |
| ECML-123-2 | | | 100% | <80% | <80% | 95% | <80% | <80% |
| ECML-183-2 | | | | 100% | <80% | <80% | <80% | <80% |
| ECML-359 | | | | | 100% | <80% | <80% | <80% |
| ECCML-363 | | | | | | 100% | <80% | <80% |
| ECML-606-1 | | | | | | | 100% | <80% |
| ECCR-664-1 | | | | | | | | 100% |

A comparison of each bacteriophage to its closest neighbor in the RefSeq Genome database is presented in Table 5. None of the E. coli phages described here were identical to previously published phages. ECML-10, ECML-123-2, ECML-183-2, ECML-363, and ECCR-664-1 were members of the same species as their closest RefSeq Genome database neighbor. ECML-1 and ECML-359 had gANI values of 93.71% and 91.83% and were considered as belonging to closely related taxa as their closest RefSeq Genome sequence. ECML-606-1 was divergent with a gANI value of <80% with Bordetella phage vB_BbrM_PHB04. However, the blast result shows only 73.33% identity over 8% query coverage suggesting that these two phages are significantly divergent. ECML-606-1 did not show any significant similarity to any of the phage genomes in the current database (accessed 12 Oct. 2021). Additionally, a blast-search against database Nucleotide collection (nr/nt) did not identify any identical phages. In summary, all these phages appear to be novel phages with significant distinction from any known and published phages.

TABLE 5
Average nucleotide identity of the deposited bacteriophages
to their closest neighbor in NCBI Reference Genome database

| Deposited | | Accession | | |
|---|---|---|---|---|
| phage | Top blast match | Number | gANI | Comments |
| ECML-1 | Escherichia phage vB_EcoM-VpaE1 | NC_027337.1 | 93.71% | Closely related taxa |
| ECML-10 | Escherichia coli bacteriophage rv5 | NC_011041.1 | 95.68% | Same species |
| ECML-123-2 | Escherichia phage vB_EcoM_DalCa | NC_054908.1 | 96.46% | Same species |
| ECML-183-2 | Escherichia phage tuntematon | NC_052660.1 | 96.74% | Same species |
| ECML-359 | Escherichia phage vB_EcoM_VR26 | NC_028957.1 | 91.83% | Closely related taxa |
| ECCML-363 | Escherichia phage wV7 | NC_019505.1 | 96.57% | Same species |
| ECML-606-1 | Bordetella phage vB_BbrM_PHB04 | NC_047861.1 | <80% | Divergent |
| ECCR-664-1 | Escherichia phage vB_EcoM_JS09 | NC_024124.2 | 96.24% | Same species |

The genome sequences of the deposited bacteriophage were scanned for motifs associated with various enzymes. Some of the results are summarized below in Table 6.

TABLE 6
Selected Motifs of Deposited Bacteriophage

| | | | | | HNH |
|---|---|---|---|---|---|
| | | Endo- | Murein | Tail | endo- |
| | Holin | lysin | hydrolase | Protein | nuclease |
| ECML-1 | Yes | Yes | No | Yes | Yes |
| ECML-10 | No | No | nudix hydrolase | Yes | Yes |
| ECML-123-2 | Yes | Yes | nudix hydrolase | Yes | Yes |
| ECML-183-2 | No | Yes | cell wall | Yes | Yes |
| | | | hydrolase | | |
| ECML-359 | Yes | No | Yes | Yes | Yes |
| ECML-363 | Yes | Yes | cell wall/ | Yes | Yes |
| | | | nudix hydrolase | | |
| ECML-606-1 | No | No | No | Yes | Yes |
| ECCR-664-1 | Yes | Yes | nudix hydrolase | Yes | Yes |

A scan of the genomic sequences of the deposited bacteriophage revealed that none of the bacteriophage in Table 6 included genes for amidase, depolymerase, ensolidase, and anti-CRISPR. All of the bacteriophages in Table 6 included genes for tail protein and HNH endonuclease. Phage tail protein are involved in recognition and binding to exterior host bacterial cell receptors and, as such, they are the primary determinants of bacteriophage's host range, i.e., how many strains of the host bacterium the phage can attach to and potentially lyse. HNH endonucleases in bacteriophages play a significant role in phage DNA packaging; phages having HNH are typically incapable of transduction which makes them well-suited for various therapeutic and biocontrol applications. Presence of both these genes in all bacteriophages in Table 6 highlights their lytic nature and strong lytic potential. The bacteriophage ECML-1, ECML-123-2, ECML-359, ECML-363, and ECCR-664-1 all have genes for holin. The bacteriophage ECML-1, ECML-123-2, ECML-183-2, ECML-363, and ECCR-664-1 all have genes for endolysin. The bacteriophage ECML-10, ECML-123-2, ECML-183-2, ECML-359, ECML-363, and ECCR-664-1 all have genes for murein hydrolase or lysin. These genes and their expression products play an important role in the lifecycle of lytic bacteriophages, and namely in the ability of bacteriophages to lyse their targeted bacterial cells. In general, bacteriophage lysis involves two different strategies. Most phages elaborate murein hydrolase (lysin) and membrane protein called holin. At the end of the lytic replication cycle, the endolysin (a peptidoglycan-degrading enzyme) is produced by phages in the host cell. They cleave the peptidoglycan cell wall, thus allowing release of progeny phage into the environment while rupturing the bacterial cell in the process. The holin, a small hydrophobic membrane spanning protein, is essential for the endolysin to translocate across the membrane to enter the periplasm. In other words, the function of the holin is to create a lesion in the cytoplasmic membrane through which the endonuclease/murein hydrolase gains access to the murein layer. The holins oligomerize to form nonspecific holes and that this hole-forming step is the regulated step in phage lysis. Presence of one or all of these genes in the Deposited Bacteriophages highlights their strong lytic potential.

### The Deposited Bacteriophages

The Deposited Bacteriophages have binding specificity for Targeted Bacteria and are capable of lysing Targeted Bacteria. The invention further contemplates variants of the Deposited Bacteriophage, which are bacteriophage having minor variation(s) in the genomic sequence and polypeptides encoded thereby while retaining the same general genotypic and phenotypic characteristics as the Deposited Bacteriophage. Such variants are considered to be the Deposited Bacteriophages in accordance with the standards advanced by Tenover for RFLP-based analysis (Tenover, Arbeit et al. 1995), or fall under the “Same Species” classification (i.e., ANI≥95%) as advanced by Olm (Olm 2017) and Jain et al. (Jain, Rodriguez et al. 2018) for full genome sequence-based analysis. The invention also contemplates progeny and bacteriophage derivative(s). The progeny, variants, substantially equivalent bacteriophages, and bacteriophage derivative(s) of the Deposited Bacteriophage all retain the same target specificity (e.g., the Targeted Bacteria) and are lytic phages.

The invention contemplates the use of the Deposited Bacteriophage, and its progeny and derivatives, to control the growth on, or colonization of, processed and unprocessed food products by Targeted Bacteria, or the colonization of buildings and equipment, particularly those associated with the processing of the same food product. The invention also provides methods of identifying Targeted Bacteria as a bacterial diagnostic and/or detecting the presence of Targeted Bacteria on processed or unprocessed food products, or equipment or buildings such as those involved in the processing of the same food products. The invention further provides methods of using the Deposited Bacteriophages for the removal of antibiotic-resistant or other undesirable pathogens from medical, veterinary, animal husbandry, or any additional environments where they may be passed to humans or animals. The invention additionally provides for methods of using the Deposited Bacteriophages to reduce the risk of, mitigate, and/or treat human and animal diseases caused by Targeted Bacteria. The Deposited Bacteriophages are administered for the methods of the invention as a homogenous phage administration, or alternatively as a component of a multi-phage composition comprising several bacteriophages, or alternatively combination of thereof with one or more prebiotic bacterial strain(s) and/or one or more probiotic yeast strain(s). These methods of use are provided with greater particularity infra. In these embodiments, the Deposited Bacteriophage may be used in combinations including two or more, three or more, four or more, five or more, six or more, or seven or more of the Deposited Bacteriophages.

Using methods and materials known in the art, a person of skill in art in possession of the Deposited Bacteriophage, will inevitably be in possession of progeny of the Deposited Bacteriophages. Indeed, after successive sub culturing of the Deposited Bacteriophages, progeny having genetic variations within the scope of “closely related” organisms are present. Furthermore, again only relaying on methods and materials known in the art, a person of skill in the art in possession of the Deposited Bacteriophage will be able to isolate and identify variants of the Deposited Bacteriophages as described herein. In particular, the variants of the Deposited Bacteriophage having minor variation(s) in the genomic sequence and polypeptides encoded thereby while retaining the same general genotypic and/or phenotypic characteristics as the Deposited Bacteriophage. Such variants are considered to be the Deposited Bacteriophage in accordance with the standards advanced by Tenover for RFLP-based analysis (Tenover, Arbeit et al. 1995), or fall under the “Same Species” classification (i.e., ANI≥95%) as advanced by Olm (Olm 2017) and Jain et al. (Jain, Rodriguez et al. 2018) for full genome sequence-based analysis. In particular, these variants may be the result of successive passaging of the Deposited Bacteriophage where the variants accumulate silent mutations, conservative mutations, minor deletions, and/or minor replications of genetic material. The variants described herein of the Deposited Bacteriophage retain the phenotypic characteristics of the Deposited Bacteriophage, in a preferred embodiment, the variants have lytic activity against the Target Bacteria.

Furthermore, bacteriophages substantially equivalent to the Deposited Bacteriophages are those bacteriophages that are “indistinguishable” from or “closely related” to the Deposited Bacteriophages under Tenover et al. (RFLP-based analysis) (Tenover, Arbeit et al. 1995) or “Same Species” under Olm (Olm 2017) and Jain et al. (Jain, Rodriguez et al. 2018) (full genome sequence-based analysis). Progeny of an organism (e.g., descendants of the organism created by serial passage of the organism), for example, are “closely related” to the parent organism, or the “Same Species” as the parent organism.

Additionally, the Deposited Bacteriophages can be used to isolate derivatives, in particular all substances that constitute subunits or expression products of the Deposited bacteriophage or its progeny, including (but not limited to) phage nucleic acids, partial or complete phage genes, gene expression products, and structural components. For example, derivatives of the invention mean lysins (also known as endolysins or murein hydrolases) produced by bacteriophages in order to cleave the cell wall of the host bacterium during the final stage of the lytic cycle. In another example, derivatives of the invention mean polyribonucleotide(s) and polydeoxyribonucleotide(s), including modified or unmodified bacteriophage DNA, cDNA, mRNA, and synthetic polynucleotide sequences, as well as DNA/RNA hybrids. Polynucleotides of the invention also encompass modified polynucleotides, such as for example phosphorylated DNAs. Depending upon the phage, the nucleic acid can be either DNA or RNA but not both and it can exist in various forms. Further, the nucleic acids of phages often contain unusual or modified bases. These modified bases protect phage nucleic acid from nucleases that break down host nucleic acids during phage infection. The size of the nucleic acid varies depending upon the phage. The phages can have only enough nucleic acid to code for 3-5 average size gene products while some phages may code for over 100 gene products.

The Targeted Bacteria—Pathogenic E. coli

E. coli encompasses an enormous population of bacteria that exhibit a very high degree of both genetic and phenotypic diversity, grouped in about 190 serogroups (Stenutz, Weintraub et al. 2006). While almost any E. coli from this enormous population can trigger human or animal disease, the subject of this invention is focused on targeting only a subset of E. coli strains (“Targeted Bacteria” or “Pathogenic E. coli”) that include (1) Shiga-toxin producing E. coli (STEC, also sometimes referred to as verocytotoxin-producing E. coli or VTEC), (2) enterohemorrhagic E. coli or EHEC, (3) enteropathogenic E. coli (EPEC), (4) enterotoxigenic E. coli (ETEC), (5) enteroaggregative E. coli (EAEC), (6) enteroinvasive E. coli (EIEC), (7) diffusely adherent E. coli (DAEC), (8) uropathogenic E. coli (UPEC), (9) meningitis-associated E. coli (MNEC) (including neonatal meningitis-causing E. coli or NMEC), (10) avian pathogenic E. coli (APEC), (11) sepsis-associated E. coli (SAEC), (12) mammary pathogenic E. coli, (13) endometrial pathogenic E. coli, (14) adherent invasive E. coli (AIEC), and (15) necrotoxigenic E. coli. For additional information about E. coli in general, and Targeted Bacteria in particular, see section “E. coli Bacteria.”

Use of the Deposited Bacteriophages and their Progeny

### Compositions

The Deposited Bacteriophage, and its progeny and derivatives, may be used to control the growth on, or colonization of, processed and unprocessed food products by Targeted Bacteria, or the colonization of buildings and equipment, particularly those associated with the processing of the same food product. The invention also provides methods of identifying Targeted Bacteria as a bacterial diagnostic and/or detecting the presence of Targeted Bacteria on processed or unprocessed food products, or equipment or buildings such as those involved in the processing of the same food products. Methods of using the Deposited Bacteriophages include for the removal of antibiotic-resistant or other undesirable pathogens from medical, veterinary, animal husbandry, or any additional environments where they may be passed to humans or animals. Methods of using the Deposited Bacteriophages to reduce the risk of, mitigate, and/or treat human and animal diseases caused by Targeted Bacteria comprise administration of an effective amount of the Deposited Bacteriophage. The Deposited Bacteriophages are administered for the methods of the invention as a homogenous phage administration, or alternatively as a component of a multi-phage composition comprising several bacteriophages. These methods of use are provided with greater particularity infra.

The Deposited Bacteriophage are formulated in compositions containing the bacteriophage and a carrier and can be stored as a concentrated aqueous solution or dried powder preparation, where dry powder preparation is obtained by convection drying, bed drying, drum drying, freeze drying (lyophilization), microwave-vacuum drying, shelf drying, electrostatic drying, infrared radiation drying, fluidized bed drying, or spray drying.

The Deposited Bacteriophage may be formulated in a chewable or gel cap or tablet composition, for example comprising gelatin, water, and the Deposited Bacteriophage, optionally including citric acid, sugar, pectin, and combinations thereof. The Deposited Bacteriophage may be formulated for oral administration with probiotic bacteria, preferably Lactobacillus species, preferably L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof. The probiotic bacteria may be included in the composition in an amount of 1-10 billion Colony Forming Units (CFU), preferably 100-10 billion CFU.

The Deposited Bacteriophage may be formulated in a chewable or gel cap or tablet composition, for example comprising gelatin, water, and the Deposited Bacteriophage, optionally including citric acid, sugar, pectin, and combinations thereof. The Deposited Bacteriophage may be formulated for oral administration with probiotic yeast, preferably Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof. The probiotic yeast may be included in the composition in an amount of 1-10 billion Colony Forming Units (CFU), preferably 100-10 billion CFU. Phages may be included in the composition in an amount of 100-one quadrillion Plague Forming Units (PFU), preferably 1,000-100 billion PFU.

The Deposited Bacteriophage may be formulated in a chewable or gel cap or tablet composition, for example comprising gelatin, water, and the Deposited Bacteriophage, optionally including citric acid, sugar, pectin, and combinations thereof. The Deposited Bacteriophage may be formulated for oral administration with probiotic bacteria, including but not limited to Lactobacillus species, preferably L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof) and probiotic yeast (preferably Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof). The probiotic bacteria may be included in the composition in an amount of 1-10 billion CFU, preferably 100-10 billion CFU; the probiotic yeast may be included in the composition in an amount of 1-10 billion CFU, preferably 100-10 billion CFU; and phage may be included in the composition in an amount of 100-one quadrillion PFU, preferably 1,000-100 billion PFU.

Bacteriophage may be formulated by resuspending purified phage preparation in aqueous medium, such as deionized water, buffer solution (e.g., Tris-HCl pH 7.4), mineral water, 5% sucrose solution, glycerol, dextran, polyethylene glycol, sorbitol, or other formulations that maintain phage viability, and are non-toxic to humans. Suitable formulations, wherein the carrier is a liquid, for administration (e.g., a nasal spray or as nasal drops, include aqueous or oily solutions of the active ingredient.) The bacteriophage may be formulated in a chewable or enteric-coated gel capsule/tablet composition comprising deionized water, buffer solution, preferably Tris-HCl pH 7.4, mineral water, 5% sucrose solution, glycerol, dextran, polyethylene glycol, sorbitol, cellulose, tapioca dextrin, hydroxypropyl methylcellulose, gellan gum, or a mixture thereof. The bacteriophage may be formulated in a chewable composition comprising polyethylene glycol, preferably PEG 3350, a sweetening agent, preferably a sugar, a polymer, preferably pectin, an organic acid, preferably citric acid, and a polyol, preferably maltitol.

A spray (including coarse spray, fine spray, mist-like spray, or fog-like spray) comprising a composition of the present invention can be produced by forcing a suspension or solution of a compound disclosed herein through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

The Deposited Bacteriophage may be formulated in pharmaceutical compositions containing the bacteriophage and a pharmaceutically acceptable carrier and can be stored as a concentrated aqueous solution or dried (e.g., spray dried or lyophilized) powder preparation. Concentrated aqueous solutions may comprise an aqueous solution with a small volume (e.g., 0.1 mL to 1 mL) and bacteriophage in an amount of about 10⁶ and 10¹¹ PFU/mL. The concentrated aqueous solution comprising a Deposited Bacteriophage may comprise the bacteriophage at about 1×10⁶ PFU/mL, 1×10⁸ PFU/mL, 1×10⁹ PFU/mL, or 1×10¹¹ PFU/mL. For example, the concentrated aqueous solution may comprise 1 mL to 10 mL of a Deposited Bacteriophage at about 1×10¹¹ PFU/mL. The aqueous solution may have a pH of pH 6.5-7.5.

The Deposited bacteriophage may be formulated as a frozen composition comprising LB broth and glycerol, e.g., 70% LB broth-30% glycerol, and stored at −20° C. to −80° C.

The Deposited bacteriophage may be formulated for oral administration by resuspending purified phage preparation in aqueous medium, such as deionized water, mineral water, 5% sucrose solution, glycerol, dextran, polyethylene glycol, sorbitol, or such other formulations that maintain phage viability, and are non-toxic to humans. Alternatively, the pharmaceutical composition can further comprise an adjuvant. The pharmaceutical composition may contain other components so long as the other components do not reduce the effectiveness of the bacteriophage so much that the therapy is negated. Pharmaceutically acceptable carriers are well known, and one skilled in the pharmaceutical art can easily select carriers suitable for particular routes of administration (Remington's Pharmaceutical Sciences, Mack Publishing Co., Easton, PA., 1985).

The pharmaceutical compositions containing Deposited Bacteriophage may be administered by parenteral (subcutaneously, intramuscularly, intravenously, intraperitoneally, intrapleurally, intravesicularly or intrathecally), topical, oral, rectal, inhalation, ocular, vaginal, optic, bladder irrigation, or nasal route, as necessitated by choice of drug and disease.

The Deposited Bacteriophage may be formulated in a pharmaceutical composition, as a dietary supplement (alone or in combination with other bacteria-based and/or yeast-based supplements), probiotic, and/or prebiotic that reduces or eliminates colonization of GI tract (including oral cavity), urinary tract, vagina, or skin with pathogenic E. coli. In effect, the Deposited Bacteriophage may be used to modulate a patient's microbiome.

The Deposited Bacteriophage may be used in a method for prophylactic treatment of a subject comprising administering the Deposited Bacteriophage to the subject in an amount sufficient to reduce pathogenic E. coli by at least 50%. In this method, the alteration of normal microflora of the individual is minimized. The subject may be a human. The Deposited Bacteriophage may be administered periodically, for example daily. The Deposited Bacteriophage can be administered in a tablet, capsule, or food or drinking additive. Additionally, a method for maintaining normal flora in a population may comprise administering the Deposited Bacteriophage to a subject in an amount sufficient to reduce pathogenic E. coli bacteria by at least 50%, whereby alteration of normal microflora is minimized. The amount administered may be an amount sufficient to eliminate pathogenic E. coli bacteria.

The invention provides a nutraceutical composition comprising at least one of the Deposited Bacteriophages, progeny, and/or variants thereof and a suitable carrier.

The Deposited Bacteriophage(s) of the invention may be administered in a powdered form in combination with additional components. The additional components can include stabilizing agents, such as salts, preservatives, bacteria-based supplements, yeast-based supplements, natural/homeopathic supplements such as cranberry juice extract and/or tea extract, and antibiotics. The additional components can also include nutritive components, such as those used to make a nutrient broth as described herein, or other useful components as determined by one skilled in the art.

The Deposited Bacteriophage may be administered in pharmaceutical compositions containing the Deposited Bacteriophage and a microgel. For example, the Deposited Bacteriophage may be encapsulated in, embedded in, crosslinked to, etc. the water soluble microgel. In some embodiments, the composition of the microgel is adapted to release the Deposited Bacteriophage in the environmental conditions found inside of a macrophage. In some embodiments, the microgel may be prepared by cross-linking polymers formed from anionic monomers and polymers formed from ionic monomers. For example, a microgel may be prepared by cross-linking poly(acrylic acid) (“PAA”) with poly(ethyleneglycol) (“PEG”) to form a poly(acrylic acid)-poly(ethyleneglycol) (PAA-PEG) microgel or hydrogel. In such embodiments, the PAA may have a molecular weight of about 25,000, the PEG may have a molecular weight of about 5,000, and the microgel may have a degree of crosslinking of about 35%. In another example, the microgel may include nanosized polymeric microgel particles including a cross-linked polymer network of polyionic segments and neutral segments as described in Vinogradov (Vinogradov 2006). Exemplary polyionic segments may be polyethylenimine (PEI) and/or PAA. Exemplary neutral segments may be PEG or Pluronic. In all embodiments, the Deposited Bacteriophage is included in 10\hat{ }6-10\hat{ }12 PFU/g of microgel, preferably 10\hat{ }7-10\hat{ }42 PFU/g microgel.

In some embodiments, the pharmaceutical compositions containing the Deposited Bacteriophage and the microgel may be adapted for macrophage-targeted delivery. In such embodiments, the microgel containing the Deposited Bacteriophage may be formed into a plurality of microgel particles that are sized to be suitable for phagocytosis by macrophages which may enhance ability of the Deposited Bacteriophages to manage infections caused by intracellular bacterial pathogens that are internalized by macrophages as part of the immune response. For example, the microgel particles may have diameters of about 1 μm to about 4 μm. In some embodiments, non-mammalian carbohydrates such as mannose, chitosan, and β-glucan may be incorporated into the microgel particles to induce phagocytosis of the microgel particles by macrophages. For example, β-glucan may be hybridized into the microgel particles. In some embodiments, the microgel particles including the Deposited Bacteriophage may be injected into a patient. In some embodiments, the microgel particles including the Deposited Bacteriophage may be intravenously administered to a patient. In some embodiments, the microgel particles including the Deposited Bacteriophage may be orally administered to a patient.

A nutraceutical composition of this invention may comprise at least one Deposited Bacteriophage in combination with an acceptable carrier. Examples of acceptable carriers include a solid, gelled, or liquid diluent, or an ingestible capsule. One or more of the bacteriophages of the invention, or a mixture thereof, may be administered orally in the form of a pill dosage form comprising the bacteriophage in combination with an acceptable carrier. A unit dosage of the bacteriophage may also be administered without a carrier material.

A nutraceutical composition comprising at least one Deposited Bacteriophage in combination with an acceptable carrier may be in the form of a capsule (e.g., enteric coated capsule), tablet, gel, syrup, or chewable composition (e.g., gummy bear). A chewable composition may comprise a binding agent, a sweetener, and at least one Deposited Bacteriophage. Pectin, food starch, gum, or any combination thereof may be used as the binding agent in the chewable composition. The chewable compositions may also include a flavoring agent, vitamins, carriers, excipients, or a combination thereof. For example, a chewable composition (e.g., gummy bear) may comprise a gummy bear mixture of sugar, glucose syrup, starch, flavoring, food coloring, citric acid, and/or gelatin, and at least one Deposited Bacteriophage. Another example of a chewable composition (e.g., gummy bear) may comprise a mixture of deionized water, buffer solution, preferably Tris-HCl pH 7.4, mineral water, 5% sucrose solution, glycerol, dextran, polyethylene glycol, sorbitol, cellulose, tapioca dextrin, hydroxypropyl methylcellulose, gellan gum, or a mixture thereof.

The nutraceutical compositions of the invention may include dietary supplements, pre-biotics, probiotics, and may be prepared in many forms that include tablets, hard or soft gelatin capsules, aqueous solutions, suspensions, and liposomes and other slow-release formulations, such as shaped polymeric gels. For example, a pill composition may comprise at least one dried Deposited Bacteriophage contained in a size “00” gel cap. An oral dosage form may be formulated such that the bacteriophage(s) of the invention are released into the intestine after passing through the stomach, to protect phage from the acidic environment (typical pH of 1.5 to 3.5) of the stomach (e.g., in enteric coated gel caps, preferably in size “0” or “00,” with the capsule capacity of 408-816 mg or 546-1,092 mg, respectively).

Oral liquid nutraceutical compositions may be in the form of, for example, aqueous or oily suspensions, solutions, emulsions, syrups, or elixirs, or may be presented as a dry product for constitution with water or other suitable vehicle before use. Such liquid compositions may contain conventional additives such as suspending agents, emulsifying agents, non-aqueous vehicles (which may include edible oils), or preservatives. For example, a gel may comprise at least one Deposited Bacteriophage.

Oral liquid nutraceutical compositions may be in the form of, for example, aqueous or oily suspensions, solutions, emulsions, syrups, or elixirs, or may be presented as a dry product for constitution with water or other suitable vehicle before use. Such liquid compositions may contain conventional additives such as suspending agents, emulsifying agents, non-aqueous vehicles (which may include edible oils), or preservatives. For example, a gel may comprise at least one Deposited Bacteriophage. In another example, an enteric capsule may comprise of at least one Deposited Bacteriophage and at least one probiotic bacteria. In yet another example, an enteric capsule may comprise of at least one Deposited Bacteriophage, at least one probiotic bacteria, and at least one probiotic yeast.

A pharmaceutical composition comprising at least one Deposited Bacteriophage in combination with a pharmaceutically acceptable carrier may be in the form of a rinse solution, capsule, tablet, gel, syrup, or chewable composition (e.g., gummy bear). A chewable composition may comprise a binding agent, a sweetener, and at least one Deposited Bacteriophage. Pectin, food starch, gum, or any combination thereof may be used as the binding agent in the chewable composition. The chewable compositions may also include a natural flavor, vitamins, carriers, excipients, or a combination thereof. For example, a chewable composition (e.g., gummy bear) may comprise a gummy bear mixture of sugar, glucose syrup, starch, flavoring, food coloring, citric acid, and/or gelatin, and at least one Deposited Bacteriophage. For example, a chewable composition (e.g., gummy bear) may comprise a mixture of deionized water, buffer solution, preferably Tris-HCl pH 7.4, mineral water, 5% sucrose solution, glycerol, dextran, polyethylene glycol, sorbitol, cellulose, tapioca dextrin, hydroxypropyl methylcellulose, gellan gum, or a mixture thereof.

The bacteriophages according to the invention may also be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the bacteriophage(s) of the invention may be in powder form, obtained by drying from solution, for constitution with a suitable vehicle, e.g., sterile saline, before use. Methods for use of bacteriophage in injectable form have been described (Merril, Biswas et al. 1996).

The bacteriophages according to the invention may also be formulated as a rinse solution for rinsing urinary tract or bladder irrigation (e.g., for reducing the risk of, mitigating, or treating UTI) and may be presented in unit dosage form in ampules, prefilled syringes, small volume infusion containers or multi-dose containers with an added preservative. The pharmaceutical compositions may take such forms as suspensions, solutions (e.g., 0.25% acetic acid solution), or emulsions in oily or aqueous vehicles, and may contain formulatory agents such as suspending, stabilizing and/or dispersing agents. The pharmaceutical compositions may further be formulated/combined with sterile saline, acetic acid, or traditional antibiotic (e.g., neomycin-polymyxin) solutions. The nutraceutical compositions may take such forms as suspensions, solutions, syrups, gel caps, capsules, tablets, and gummy bears that may contain formulatory agents such as suspending, stabilizing and/or dispersing agents. The nutraceutical compositions may be further formulated/combined with one or more of other (e.g., natural/homeopathic) ingredients that enhance prophylactic or therapeutic effect of deposited bacteriophages in managing UTI infections. In exemplary embodiments, the nutraceutical composition is combined with proanthocyanidin, and/or cranberry juice extract, and/or D-mannose, and/or vitamins (preferably vitamin C), and/or acetic acid, and/or celery extract to reduce the risk of, mitigate, or treat urinary tract infections caused by UPEC whereas the composition is administered orally or as a urinary tract rinse. Alternatively, the bacteriophage(s) of the invention may be in powder form, obtained by drying from solution, for constitution with a suitable vehicle, e.g., sterile saline, before use.

For topical administration to the epidermis, the bacteriophage(s) may be formulated as rinse solutions, ointments, creams, or lotions. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Pharmaceutical compositions or nutraceutical compositions suitable for topical administration in the mouth include unit dosage forms such as lozenges comprising a bacteriophage(s) of the invention in a flavored base, usually sucrose and acadia or tragacanth. Pastilles comprising one or more bacteriophages in an inert base such as gelatin and glycerin or sucrose and acacia are also provided. Mucoadherent gels and mouthwashes comprising a bacteriophage(s) of the invention in a suitable liquid carrier are additionally provided.

The present invention relates to stabilized bacteriophage formulations and their use as delivery systems. More particularly, the present invention pertains to stabilized bacteriophage formulations, methods for preparing stabilized bacteriophage formulations and uses of stabilized bacteriophage formulations. For example, a pharmaceutical composition or nutraceutical composition may comprise at least one of the Deposited Bacteriophages and a water-soluble polymer and sugar, derivatives of cellulose, or polyvinylpyrrolidone low or medium molecular, or glycols with a molecular weight of 4000 or 6000, or sodium alginate, and sugars—trehalose and/or maltodextrin and/or lactose and/or mannitol as cellulose derivatives used sodium salt of carboxymethylcellulose, or a mixture thereof.

The present invention provides a method for producing a composition comprising, adsorbing an aqueous solution of bacteriophages, or phage components, onto a solid or powdered matrix to produce composition, and drying the composition to produce a composition, when drying is accomplished by convection drying, bed drying, drum drying, freeze drying (lyophilization), microwave-vacuum drying, shelf drying, infrared radiation drying, electrostatic drying, fluidized bed drying, or spray drying.

The present invention also pertains to the method described above wherein the matrix may be selected from the group consisting of skim milk powder, soya protein powder, whey protein powder, albumin powder, casein, gelatin, single cell proteins, algal protein, plant peptone, trehalose, maltodextrin, mannitol, powdered sugar, sugar alcohol, charcoal, latex beads, a water-soluble carbohydrate-based material, talc, chitin, and fish cartilage.

The present invention also provides a nutraceutical composition comprising at least one Deposited Bacteriophage, or phage component, adsorbed onto a matrix. For example, a nutraceutical composition comprising at least one Deposited Bacteriophage, or phage component, adsorbed onto dry animal feed for delivering effective dose of the Deposited Bacteriophage or phage component to animals in need of reducing the risk of, mitigating, or treating disease caused by the Targeted Bacteria.

The present invention also provides a pharmaceutical composition comprising at least one Deposited Bacteriophage, or phage component, adsorbed onto a matrix. For example, a pharmaceutical composition comprising at least one Deposited Bacteriophage, or phage component, adsorbed onto dry animal feed for delivering effective dose of the Deposited Bacteriophage or phage component to animals in need of reducing the risk of, mitigating, or treating disease caused by the Targeted Bacteria.

The present invention includes the material as defined above, wherein the soluble matrix is selected from the group consisting of dry animal feed, skim milk powder, soya protein, albumin powder, single cell proteins, trehalose, mannitol, sugar, and sugar alcohol.

The compositions of the present invention are easy to prepare and exhibit the property of being stable over various lengths of time at refrigerator and room temperatures, from about −10° C. to about 25° C.

Compositions of the present invention with little or no loss in titer. The antibacterial compositions of the present invention may be used within lotions, lubricants, gels and creams, suppositories, toothpaste, be admixed with a pharmaceutically acceptable carrier for oral, nasal, or topical applications for example but not limited to skin, vaginal, ophthalmic, nasal, aural, urinary, anal, rectal, and other types of administration, or be used within wound dressings, and exhibit antimicrobial activity.

The present invention provides stabilized phage preparations in a dry form as a storage and/or delivery system for powder formulations. The present invention also provides a suitable matrix for preparing phage or phage compositions for encapsulation or mixing with dry animal feed and delivery to the animal gut past the stomach acids.

The present invention provides stabilized phage preparations in a dry form as a delivery system for powder inhalants. The present invention also provides a suitable matrix for preparing phage or phage compositions for encapsulation and delivery to the human gut past the stomach acids.

Pharmaceutical compositions or nutraceutical compositions suitable for rectal administration are most preferably presented as unit dose suppositories. Suitable carriers include saline solution, nutrient broths, and other materials commonly used in the art. Compositions suitable for vaginal administration may be presented as pessaries, tampons, creams, gels, pastes, foams, or sprays that contain a carrier in addition to a bacteriophage. Such carriers are well known in the art.

For administration by inhalation, the bacteriophage(s) according to the invention are conveniently delivered from an insufflator, nebulizer, or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the bacteriophage(s) of the invention may take the form of a dry powder composition, for example, a powder mix of the bacteriophage(s) and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, the bacteriophage(s) of the invention may be administered via a liquid spray, such as via a plastic bottle atomizer. For topical administration to the eye, the bacteriophage(s) according to the invention can be administered as drops and gels. For urinary tract rinse/bladder irrigation, the bacteriophage(s) according to the invention can be administered as rinses in a suitable carrier such as sterile saline, acetic acid (e.g., 0.25% acetic acid solution), antibiotic (e.g., neomycin-polymyxin solution), or combination thereof.

Pharmaceutical compositions or nutraceutical compositions of the invention may also contain other adjuvants such as flavorings, colorings, anti-microbial agents, or preservatives. The invention also provides kits containing packaging and a bacteriophage(s) of the invention.

Dose and duration of therapy will depend on a variety of factors, including the patient age, patient weight, and tolerance of the phage. Bacteriophage may be administered to patients in need of the therapy provided by this invention by oral administration. Based on previous human experience in the former Soviet Union and Europe, a dose of phage between 10⁶ and 10¹¹ PFU will be suitable in most instances (Sulakvelidze and Kutter 2005). For example, the bacteriophage may be present in a composition in an amount between 10⁶ and 10¹² PFU. The bacteriophage may be present in a composition in an amount about 10⁶, 10⁷, 10⁸, 10⁹, 10¹⁰, 10¹¹, or 10¹² PFU. The bacteriophage may be present in a composition in an amount between 10⁶ and 10⁸, 10⁶ and 10⁹, 10⁶ and 10¹⁰, or 10⁷ and 10¹² PFU. The phage may be administered orally in, for example, mineral water, optionally with 1.0-3.0 grams of sodium bicarbonate added to reduce stomach acidity. Alternatively, sodium bicarbonate may be administered separately to the patient just prior to dosing with the phage. Phages also may be incorporated in a tablet or capsule which will enable transfer of phages through the stomach with no or little reduction of phage viability due to gastric acidity, and release of active phages in the small intestine (with potential additional protection against bile salts). The frequency of dosing will vary depending on how well the phage is tolerated by the patient and how effective a single versus multiple doses is at reducing pathogenic E. coli gastrointestinal colonization.

The dose of Deposited Bacteriophage and duration of therapy for a particular patient can be determined by the skilled clinician using standard pharmacological approaches in view of the above factors. The response to treatment may be monitored by, analysis of blood, fecal matters, or body fluid levels of pathogenic E. coli, or pathogenic E. coli levels in relevant tissues or monitoring disease state in the patient. The skilled clinician will adjust the dose and duration of therapy based on the response to treatment revealed by these measurements.

One of the major concerns about the use of phages in clinical settings is the possible development of bacterial resistance against them. However, as with antimicrobial resistance, the development of resistance to phages takes time. The successful use of phages in clinical settings will require continual monitoring for the development of resistance, and, when resistance appears, the substitution of other phages to which the bacterial mutants are not resistant. In general, phage preparations may be constructed by mixing several separately grown and well-characterized lytic phages, in order to (i) achieve the desired, broad target activity of the phage preparation, (ii) ensure that the preparation has stable lytic properties, and (iii) minimize the development of resistance against the preparation. The invention provides for a method of formulating phage preparations comprising of one or more of Deposited Bacteriophage to reduce the frequency of bacterial resistance against phages.

The invention also provides for a method for modulating an animal's microbiome by preventing colonization or reducing colonization by pathogenic E. coli comprising administration of an effective amount of a composition comprising at least one of the isolated bacteriophage ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249) deposited to the ATCC, said bacteriophage having lytic activity against pathogenic E. coli strains, and variants thereof, wherein said variants retain the phenotypic characteristics of said bacteriophage and wherein said bacteriophage and variants thereof have lytic activity against pathogenic E. coli strains. In this embodiment, the Deposited Bacteriophage may be used in combinations including two or more, three or more, four or more, five or more, six or more, or seven or more of the Deposited Bacteriophages. The composition may be a pharmaceutical composition or nutraceutical composition including dietary supplement, probiotic, and/or prebiotic. The composition may be formulated as a capsule, tablet, suppository, chewable composition, syrup, or gel. The capsule may be a gel capsule, preferably enteric coated gel capsule. In a method for modulating an animal's microbiome by preventing or reducing colonization by pathogenic E. coli. In animals, animal species may be alpacas, armadillos, capybaras, cats, chimpanzees, chinchillas, cattle, dogs, goats, gorillas, horses, humans, lemurs, llamas, mice, non-human primates, pigs, rats, sheep, shrews, and tapirs. In avian animals, avian species may be poultry (chickens, turkeys, geese, and ducks), wildlife, waterfowl, psittacine birds, and game birds. In humans, the patient may be an adult, infant, or child, for example, a child is less than 5 years of age. The pathogenic E. coli strain may be STEC, EHEC, EPEC, ETEC, EAEC, EIEC, DAEC, UPEC, MNEC, avian pathogenic E. coli, sepsis-associated E. coli, mammary pathogenic E. coli, endometrial pathogenic E. coli, necrotoxigenic E. coli, or a combination thereof.

In a method for modulating an animal's microbiome by reducing colonization by pathogenic E. coli bacteria strains the patient may be colonized by a pathogenic E. coli strain or strains. The bacteriophage may be present in the pharmaceutical composition or nutraceutical composition in an amount of 10⁶ and 10¹² PFU. The method may reduce pathogenic E. coli colonization of the gastrointestinal tract, vagina, urinary tract, skin, or a combination thereof.

The development of neutralizing antibodies against a specific phage is possible. However, the development of neutralizing antibodies may not pose a significant obstacle in the proposed clinical settings, because the kinetics of phage action is much faster than is the host production of neutralizing antibodies. For example, phages can be used for a few days (e.g., 1-14 days), sufficient to reduce bacterial colonization during the time when immunocompromised patients are most susceptible to the development of potentially fatal septicemia caused by pathogenic E. coli, but not long enough for phage-neutralizing antibodies to develop in sufficient concentrations to hinder the treatment efficacy. If the development of anti-phage antibodies is a problem, several strategies can be used to address this issue. For example, different combination of one or more of the Deposited Bacteriophages may be administered at different times during therapy. It will be appreciated that the amount of the present bacteriophages, required for use in treatment will vary not only with the particular carrier selected but also with the route of administration, the nature of the condition being treated and the age and condition of the patient. Ultimately the attendant health care provider may determine proper dosage.

### Food Preservation

In one embodiment, the invention contemplates a method for reducing the risk of foodborne illnesses or spoilage caused by the Targeted Bacteria, comprising contacting a food product or products intended for humans or animals with a microbial growth inhibiting effective amount of a bacteriophage composition comprising the Deposited Bacteriophage. The modes of contact include, but are not limited to, spraying, misting, or fogging the Deposited Bacteriophages composition on the food product(s), or by dipping or soaking the food product(s) in a solution containing a concentration of the Deposited Bacteriophages sufficiently high to inhibit the growth of Targeted Bacteria, or adding, injecting, or inserting the Deposited Bacteriophages into the food product(s) in a said concentration.

In another embodiment, the invention contemplates the application of the Deposited Bacteriophages composition to equipment associated with the processing of food product(s), such as cutting instruments, conveyor belts, and any other implements utilized in the production of food products, including the preparation, storage and packaging steps of food processing. The Deposited Bacteriophages can additionally be introduced into packaging materials used to contain food product(s), prior to or following transfer of the food product(s) to the packaging materials. Alternatively, the Deposited Bacteriophages can be useful in the local processing of food products located, for example, in the home or in a restaurant kitchen, using the same modes of contact as described supra.

In another embodiment of the invention, the Deposited Bacteriophages are added as a component of paper products, either during processing or after completion of processing of the paper products. Paper products to which the Deposited Bacteriophages may be added include, but are not limited to, paper towels, toilet paper, moist paper wipes. In a preferred embodiment of the invention, the Deposited Bacteriophages are added as a component of cleansing wipes. The Deposited Bacteriophages may be added in an aqueous state to a liquid-saturated paper product, or alternatively may be added in powder form, to dry paper products, or any combination thereof. In similar manner, the Deposited Bacteriophages may be incorporated into films such as those used for packaging foods, such as by impregnating or coating the film or plastic or paper containers or bags used for storing or transporting food products.

The methods of the invention further contemplate the application of the Deposited Bacteriophages to the floors, walls, ceilings, drains, conveyer belts, cutting surfaces, or other environmental surfaces in structures such as the industrial food processing, military, or home environments. In a particularly preferred embodiment of the invention, the Deposited Bacteriophages are applied to refrigerated devices used to store or transport food or food products, including but not limited to, home and industrial refrigerators, deli-meat and cheese counters, refrigerated trucks, and mobile food-service vehicles.

In a non-limiting embodiment of the invention, the Deposited Bacteriophages of the invention are useful in preventing the colonization of, or inhibiting the growth of, Targeted Bacteria on processed or unprocessed food products by infecting, lysing, or inactivating Targeted Bacteria present on said food product. Processed or unprocessed food products intended for humans in which the Deposited Bacteriophages are particularly useful in preventing the growth or colonization of Targeted Bacteria include, but are not limited to, poultry and beef (particularly ground poultry and beef), fresh vegetables exposed to Targeted Bacteria such as lettuce, spinach, green onions, and other fresh fruits and vegetables commonly grown out of doors in fields. Processed or unprocessed food products intended for animals in which the Deposited Bacteriophages are particularly useful include wet pet foods, moist pet foods, and dry pet foods intended for household pets, as well as feed intended for domesticated animals such as horses, cows, sheep, pigs, chickens, turkeys, and fish raised in farming or aquaculture environments.

The Deposited Bacteriophages can also be administered to potable and non-potable water sources to reduce or eliminate the presence of Targeted Bacteria.

Bacteriophage compositions of the invention may be provided in aqueous or non-aqueous embodiments for the preservation of food.

Aqueous embodiments of the Deposited Bacteriophages include aqueous compositions comprising, or alternatively consisting of, one of the Deposited Bacteriophages alone or in combination with other Deposited Bacteriophages, or with another bacteriophage or other bacteriophages. Aqueous embodiments of the Deposited Bacteriophages are available in solutions that include, but are not limited to, phosphate buffered saline, Luria-Bertani Broth, or water with the levels of chlorine less than 10 ppm.

Non-aqueous embodiments of the Deposited Bacteriophages include, but are not limited to, dried compositions comprising, or alternatively consisting of, the Deposited Bacteriophages alone or in combination with other bacteriophage(s). Freeze-dried and spray-dried compositions may also include soluble and/or insoluble carrier materials as, for example, processing aids.

The Deposited Bacteriophages can be administered at a concentration effective to prevent the initial colonization of foods with Targeted Bacteria, or to inhibit the growth or colonization of food or food products, as well as the equipment used to process or store food. In a non-limiting embodiment of the invention, the Deposited Bacteriophages typically administered at a growth inhibiting effective amount of a concentration of about 10⁷ to about 10¹¹ Plaque Forming Units (PFU)/mL. One of skill in the art is capable of ascertaining bacteriophage concentrations using widely known bacteriophage assay techniques (Adams 1959). The Deposited Bacteriophages at such concentrations may be applied at, for example, about 1-4 mL/500 cm² of food surface or 10⁶-10¹⁰ PFU/g food product.

### Food Processing Uses

The present invention provides a method for reducing the risk of growth of microorganisms on food products comprising contacting a food product with an effective amount of a composition comprising at least one of the Deposited Bacteriophage for reducing the risk of growth of pathogenic E. coli microorganisms on food products. Reducing the risk of growth of microorganisms on food products is intended to provide a food product that is devoid of or contains minimal numbers of viable microorganisms that could cause illness in humans or animals or spoilage of the food product prior to ingestion. The food product may be fruit juices, vegetable juices, produce (including fruits, vegetables, grains, and oats), wheat kernels, flour, seafood and selfish, poultry, beef, lamb, or pork.

Reducing the risk of growth of microorganisms on food products is intended to include but is not limited to the following mechanisms: (1) removal of attached microorganisms from the food products; (2) inhibition of attachment of microorganisms to the food products; (3) killing or inactivation of attached microorganisms on the food products; and (4) killing or inactivation of microorganisms which are not attached to the food product but which are present in liquids associated with the food products during processing; such as in chill tanks, or which are present on surfaces associated with food preparation, liquids remaining on such surfaces, such as countertops, cutting boards and sinks, and equipment used in food preparation and sanitization of the food.

The present invention has an important application in the food processing industry, as well as for home and institutional food preparation. The Deposited Bacteriophage compositions of the invention are readily available and the cost of carrying out the method of the present invention is not expensive as compared to some of the existing antimicrobial processes. Unlike existing treatments using, for example, trisodium phosphate or irradiation, the use of the Deposited Bacteriophage compositions of the invention does not alter the appearance, color, taste, or texture of the food product. Moreover, the Deposited Bacteriophage compositions of the invention are non-toxic. The Deposited Bacteriophage compositions may be readily applied to food processing equipment and food processing workspaces. For example, a composition comprising the Deposited Bacteriophage may be applied by spraying onto a surface or equipment used in food processing. The Deposited Bacteriophage compositions may be readily applied to food preparation equipment and food preparation workspaces, e.g., surfaces used in food preparation work.

The Deposited Bacteriophage composition is applied for a period of time sufficient to kill pathogenic E. coli bacteria present on the food product. It is important that the application time of the Deposited Bacteriophage compositions is for a sufficient time to result in significant reduction of the risk of growth of pathogenic E. coli on the food product.

The present invention also includes methods of contacting the Deposited Bacteriophage compositions of the invention with food products, including but not limited to, spraying or misting or fogging the compound on the food product, or by immersing the food product in a composition comprising at least one of the Deposited Bacteriophages of the invention.

The present invention is intended to encompass any method that contacts the Deposited Bacteriophage compositions of the invention with a food product by any direct means, including spraying, misting, fogging, dipping, or soaking. But the present invention also is intended to include contact of the Deposited Bacteriophage compositions of the invention with the food by indirect means, such as applying the Deposited Bacteriophage compositions of the invention to equipment or food product processing or preparation surfaces in which the food product is contacted during processing, preparation, storage, and/or packaging.

Any type of method of contact of the Deposited Bacteriophage compositions with the food product is preferred as long as it is capable of allowing a short application time while ensuring even and thorough coverage of the treatment area: A method that utilizes a cabinet that provides spraying or misting or fogging of the food product is useful in the present invention. Machinery for use in such cabinets on a processing line in a food processing plant are adaptable for reducing the application time to a minimum while still obtaining efficacious antimicrobial effects on the food.

The present method is useful, for example, in a poultry processing plant for treating post-chilled chickens that have been immersed in a chill bath of cold water. The chickens are removed from the chill bath and treated with the Deposited Bacteriophage compositions of the invention for an application time sufficient to result in significant reduction of the risk of growth of microorganisms on the chickens. The treated chickens are subsequently packaged without further washing or rinsing. However, the method optionally may include, if deemed necessary, at least one washing step of the chickens prior to packaging. The optional washing step may include spraying or misting the food product with water or immersing the food product in a container or tank of water.

Further, the method of the present invention can optionally include a determination step prior to contacting the food product with the Deposited Bacteriophage compositions of the invention to determine the presence of microorganisms on the food before treatment. Any conventional methods for rapidly determining the presence of microorganisms can be utilized as the determination step, which for example, includes PCR and immunoassays.

Further, the method of the present invention can optionally include a determination step to select the Deposited Bacteriophage compositions that are most effective in reducing or eliminating pathogenic E. coli in the food product. For example, pathogenic E. coli strains could be screened for their susceptibility to each of the Deposited Bacteriophages by the drop-on-lawn method, also known as the “spot test” method, essentially as described in (Adams 1959) once the testing results are available, the Deposited Bacteriophages most effective in lysing the targeted pathogenic E. coli strains could be selected and formulated into the Deposited Bacteriophage composition that is most effective in reducing or eliminating pathogenic E. coli in the food product.

Additionally, the method of the present invention optionally includes a step to determine the presence of the bacteriophage compositions of the invention on the surface of the food product after contact with the Deposited Bacteriophage compositions. This determination is performed immediately after the contacting step or after several washing steps. For example, the Deposited Bacteriophage compositions of the invention is extracted from the tissues of the food in a form suitable for high performance liquid chromatography (HPLC) analysis or PCR analysis or direct plating analysis.

The food processing industry, as well as home, restaurant, or institutional food preparation, needs more effective products and processes for the reduction of the risk of growth of a broad range of contaminating microorganisms on many different food products and/or surfaces that the food products and juices or liquids from the food come in contact. This is especially true for microorganisms which are attached to the surfaces of food. As a result of increasing numbers of illnesses caused by foodborne pathogenic microorganisms, the food processing industry now requires more effective processes for the removal and reduction of the risk of growth of a broader spectrum of microorganisms, and particularly for pathogenic microorganisms, such as pathogenic E. coli, which are known to cause serious human diseases because of food contamination. The present invention provides a composition comprising at least one Deposited Bacteriophages of the invention and methods of reducing the risk of growth of microorganisms on and in the food, as well as in liquids and on surfaces associated with food products and their preparation. This method of reducing the risk of growth of microorganisms is an important goal in reducing the risk of cross-contamination from infected food products; in removing attached microorganisms from food products; in inhibiting the attachment of microorganisms to the food products; and in reducing the risk of growth of microorganisms that remain attached to the food products. Further, the method of the present invention can easily be adapted for use in a food processing plant.

### Environmental Control

In another embodiment of the invention, the Deposited Bacteriophages are administered to environments to control the growth or viability of Targeted Bacteria. Environments in which the Deposited Bacteriophages are useful to control the growth or viability of Targeted Bacteria include, but are not limited to, abattoirs, meat processing facilities, feedlots, vegetable processing facilities, medical facilities (including hospitals, out-patient clinics, school and/or university infirmaries, and doctors' offices), military facilities, veterinary offices, animal husbandry facilities, public and private restrooms, and nursing and nursing home facilities. The invention further contemplates the use of the Deposited Bacteriophages for the battlefield decontamination of food stuffs, the environment, and personnel and equipment, both military and non-military.

The Deposited Bacteriophages are additionally useful alone or in combination with other bacteriophage(s) and/or other compounds (e.g., exopolysaccharide-degrading enzymes encoded by the phage genomes), for reducing the risk of formation of biofilms, or controlling the growth of biofilms, in various environments. Aqueous embodiments of the Deposited Bacteriophages are available in solutions that include, but are not limited to, phosphate buffered saline, Luria-Bertani Broth, or chlorine-free water (water that contains less than 10 ppm chlorine). In a particularly preferred embodiment, the Deposited Bacteriophages are used to control biofilm formation and growth in municipal water systems, industrial water systems, and personal water systems, as well as biofilms present in refrigerated environments.

The modes of administration include, but are not limited to, spraying, moping, hosing, and any other reasonable means of dispersing aqueous or non-aqueous bacteriophage compositions, in an amount sufficiently high to inhibit the growth or viability of Targeted Bacteria. In a non-limiting embodiment of the invention, the Deposited Bacteriophages are useful in reducing the risk of growth or reducing viability of Targeted Bacteria by infecting, lysing, or inactivating Targeted Bacteria present in said environment. Administration of the Deposited Bacteriophages composition includes application to the floors, walls, countertops, ceilings, drains or any other environmental surface.

Bacteriophage compositions of the invention are available in aqueous or non-aqueous embodiments discussed earlier for Food Preservation applications.

In another embodiment of the invention, the Deposited Bacteriophages are added as a component of paper products, either during processing or after completion of processing of the paper products. Paper products to which the Deposited Bacteriophages may be added include, but are not limited to, paper towels, toilet paper, and moist paper wipes. In a preferred embodiment of the invention, the Deposited Bacteriophages are added as a component of cleansing wipes; it may be added in an aqueous state to a liquid-saturated paper product, or alternatively may be added in powder form such as a spray-dried preparation, to dry paper products, or any combination thereof.

The Deposited Bacteriophages can be administered at a concentration effective to inhibit the growth or viability of Targeted Bacteria in a particular environment. In a non-limiting embodiment of the invention, the Deposited Bacteriophages are administered at a concentration of about 10⁷ to 10¹² PFU/mL. In another non-limiting embodiment of the invention, the Deposited Bacteriophages are administered at a concentration of about 10-100 mL/500 cm 2 of environmental surface. One of skill in the art is capable of ascertaining bacteriophage concentrations using widely known bacteriophage assay techniques (Adams 1959).

### Nutraceutical Uses (Dietary Supplements, Prebiotics, or Probiotics)

The Deposited Bacteriophages may be formulated into nutraceutical compositions, which could include one or more of the Deposited Bacteriophages and be in the form of dietary supplements, prebiotics, or probiotics.

In addition to one or more of the Deposited Bacteriophages, the nutraceutical compositions may also include one or more probiotic bacterial strains, preferably Lactobacillus species, preferably L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof.

In addition to one or more of the Deposited Bacteriophages, the nutraceutical compositions may also include one or more probiotic yeast strains, preferably Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof.

In addition to one or more of the Deposited Bacteriophages, the nutraceutical compositions may also include one or more of other ingredients that enhance prophylactic or therapeutic effect of deposited bacteriophages, including but not limited to proanthocyanidin, cranberry juice extract, D-mannose, vitamins (preferably vitamin C), acetic acid, and celery extract.

In addition to one or more of the Deposited Bacteriophages, the nutraceutical compositions may also include one or more aforementioned probiotic bacterial strains and yeast strains.

The nutraceutical compositions may be administered to a pet, agriculturally important animal, or human patient, wherein the Deposited Bacteriophages lyse the Targeted Bacteria. This lysis of the Targeted Bacteria may lead to a better microflora balance and confer a health benefit on the recipient. Combination of the Deposited Bacteriophages and aforementioned probiotic bacterial strains and/or yeast strains may provide enhanced health benefits. For example, lysis of the Targeted Bacteria by the Deposited Bacteriophages may reduce diarrhea caused by pathogenic E. coli, whereas Saccharomyces boulardii may concurrently help alleviate other general digestion problems, e.g., irritable bowel syndrome. In another example, lysis of the Targeted Bacteria by the Deposited Bacteriophages may mitigate, reduce the risk of, or treat urinary tract infections by reducing UPEC levels in the urinary tract, whereas cranberry juice extract and/or D-mannose may concurrently help reduce the risk of or mitigate UPEC adherence to surface along the urinary tract thereby enhancing lytic effect of the Deposited Bacteriophages.

### Reduction of the Risk of, Mitigation, or Treatment of Infection or Prevention or Treatment of Colonization

In another embodiment, the invention contemplates a method for the reduction of the risk of, mitigation or treatment of illnesses caused by the Targeted Bacteria, comprising contacting a microbial growth inhibiting effective amount of a bacteriophage composition comprising the Deposited Bacteriophages with a site or sites of infection of a host mammal infected with Targeted Bacteria.

At the time bacteriophages were discovered, with the age of antibiotics still in the future, bacteriophages were considered to be a potentially powerful cure for bacterial infections, and they were therapeutically utilized throughout the world during the pre-antibiotic era. The use of phages in humans was found to be very safe; however, phage therapy did not always work and, with the advent of antibiotics that were effective against a broad spectrum of pathogenic bacteria, it gradually fell out of favor in the United States and Western Europe. Several factors, including the lack of a broad understanding of phage biology, the “Soviet Taint,” and inadequate diagnostic bacteriology techniques, contributed to the failure of some of the early phage therapy studies and to the associated decline of interest in phage therapy in the West. Reviewed in more detail in (Sulakvelidze and Kutter 2005). At the same time, phage therapy continued to be utilized in the former Soviet Union and Eastern Europe, where phage therapy still is being used to treat a wide range of bacterial diseases ranging from intestinal infections to septicemia. Comprehensive information about human and veterinary applications of bacteriophages has been recently reviewed by several investigators. See (Alisky, Iczkowski et al. 1998), (Sulakvelidze and Kutter 2005).

The infected mammalian host may be a human host or animal host. In particular, the host may be a bovine, poultry, or porcine host. Reducing the risk of developing the infection by Targeted Bacteria, or treatment of infected persons or animals, is particularly preferred in immuno-compromised persons, pregnant females, and newborns and infants, who maybe at an elevated risk of infection by Targeted Bacteria. The modes of contact include, but are not limited to, spraying, or misting the bacteriophage composition on the infected mammalian host, by injecting at a site or sites of infection a pharmaceutically acceptable composition containing a concentration of the Deposited Bacteriophages sufficiently high to inhibit the growth of Targeted Bacteria, or by ingesting a solution containing a concentration of the Deposited Bacteriophages sufficiently high to inhibit the growth of Targeted Bacteria. Additional routes of administration include but are not limited to oral, rectal, topical, ophthalmic, buccal, intravenous, otic, nasal, vaginal, inhalation, and intrapleural.

In another nonlimiting embodiment of the invention, the Deposited Bacteriophages are useful for preparing bacterial vaccines or bacterins that eliminate or reduce colonization of the Targeted Bacteria in, and/or their being shed by various agriculturally important animals. One example of a practical application for that type of vaccine is in the poultry-raising industry, where its administration may significantly reduce colonization of poultry with the Targeted Bacteria; thus, improving public safety by reducing contamination of poultry with the Targeted Bacteria.

In yet another nonlimiting embodiment of the invention, the Deposited Bacteriophages are useful for preparing bacterial vaccines or bacterins that eliminate or reduce colonization of the Targeted Bacteria in, and/or their being shed by, various agriculturally important animals. One example of a practical application for that type of vaccine or bacterin is in the poultry-raising industry, where its administration may significantly reduce colonization of poultry with the Targeted Bacteria; thus, improving safety of the animals and enhancing their growth dynamics (e.g., improving feed conversion ratios in birds) by reducing or eliminating colonization of poultry with the Targeted Bacteria.

Bacteriophage compositions of the invention are available in aqueous or non-aqueous embodiments discussed earlier for Food Preservation applications.

The Deposited Bacteriophages can be administered at a concentration effective to inhibit the growth or viability of Targeted Bacteria in the infected host. In a non-limiting embodiment of the invention, the Deposited Bacteriophages are administered at a concentration of about 10⁷ to 10¹¹ PFU/mL. In another non-limiting embodiment of the invention, the Deposited Bacteriophages are administered at a concentration of about 1-4 mL/500 cm² of food surface or 10⁶-10¹⁰ PFU/g food product. One of skill in the art is capable of ascertaining bacteriophage concentrations using widely known bacteriophage assay techniques (Adams 1959).

Depending on the severity of peculiarities of the infection, the Deposited Bacteriophages can be administered to animals (including humans) (i) orally, in tablet or liquid formulation (10⁶-10¹² PFU/dose), (ii) rectally, (iii) locally (skin, eye, ear, nasal mucosa, etc.), in tampons, rinses and creams, (iv) as aerosols or intrapleural injections, (v) rinses (e.g., for bladder irrigation), and (vi) intravenously.

### Use of Bacteriophage Derivatives

Derivatives, such as polypeptides, including but not limited to bacteriophage lytic enzymes, encoded by the bacteriophage or the bacteriophage progeny are used for applications designed to reduce the risk of growth of Targeted Bacteria through cell wall lysis. In this context, lytic polypeptides are useful for reducing the risk of growth of Targeted Bacteria on processed and unprocessed food products, as well as equipment used for the processing of said food products.

In another preferred embodiment of the invention, bacteriophage derivatives are useful for the treatment of one or more infections in a mammal, including humans, by administering their therapeutically effective amounts to the patient. This method is useful for the treatment of infections of the gastrointestinal system. Similarly, this method is useful in a prophylactic setting for reducing the risk of infection by Targeted Bacteria in pregnant mammals, including humans. This method of treatment is further useful for reducing the risk of other disorders, or infections caused by Targeted Bacteria, such as acute bloody or non-bloody diarrhea, sometimes associated with hemolytic-uremic syndrome.

Another nonlimiting embodiment of the invention is that the bacteriophage derivatives such as lysins will be useful for preparing bacterial vaccines or bacterins that eliminate or reduce colonization of the Targeted Bacteria in, and/or their being shed by various agriculturally important animals. One example of a practical application for that type of vaccine is in the cattle-raising industry, where administration of such vaccines/bacterins may significantly reduce colonization of cattle with the Targeted Bacteria; thus, improving public safety by reducing contamination of beef with the Targeted Bacteria.

### Detection Systems

The Deposited Bacteriophage, its progeny, recombinant bacteriophage, or derivatives of the above are useful in methods of screening environmental samples (including food products and food processing equipment) and clinical specimens for the presence of viable cells of Targeted Bacteria. For example, in one such system, recombinant bacteriophage containing a reporter system such as, for example, a luciferase reporter system is applied to the sample and analyzed at some time point in the future for the activation of the reporter molecule. The activation of the reporter molecule is indicative of the presence of viable cells of Targeted Bacteria.

The Deposited Bacteriophage, their progeny, recombinant bacteriophage, or derivatives such as lytic enzymes are useful in methods of screening environmental samples including food products and food processing equipment and clinical specimens for the presence of viable cells of Targeted Bacteria, by monitoring and measuring bacterial metabolism products such as bacterial adenosine kinase (AK) or adenosine triphosphate (ATP) released as a result of specific lysis of Targeted Bacteria. For example, when the released ATP is incubated with a luciferin/luciferase mixture, a rapid flash of peak light emission occurs within less than a second, followed by a steady glow lasting for several hours. By measuring the luminescence, it is possible to obtain a quantitative estimate of the number of bacterial cells in a sample. Although the basic approach involved in such detection-based assays is well-established, the existing assays have shortcomings that hinder their wide acceptance. For example, the various reagents that have been used to lyse bacteria and release their ATP have broad-specificity; therefore, ATP is released from all susceptible bacterial and eukaryotic cells present in the sample, which can cause false-positive readings. In this context, the original Deposited Bacteriophage, its progeny, recombinant bacteriophage, or derivatives such as lytic enzymes will specifically lyse Targeted Bacteria without affecting any other prokaryotic or eukaryotic cells that may be present in the sample, thus providing means for accurately and specifically identifying and detecting Targeted Bacteria.

### Epidemiological Typing

The Deposited Bacteriophage, and/or their progeny and derivatives may be further useful as a tool for the epidemiological typing of Targeted Bacteria. For example, one of skill in the art can use the Deposited Bacteriophages of the invention to screen a panel of Targeted Bacteria isolates to aid in the taxonomic identification of the Targeted Bacteria, by determining which isolates yield a positive lytic reaction to the Deposited bacteriophage. For example, see (van der Mee-Marquet, Loessner et al. 1997).

### Preparation of Vaccines or Bacterins

The Deposited Bacteriophage, and/or its progeny and derivatives, also may be valuable for preparing bacterial lysates to be used as vaccines or bacterins. The immunogenicity of such vaccines or bacterins may be superior to that of so-called dead cell vaccines because phage-mediated lysis is a more effective and gentler approach for exposing protective antigens of bacteria than are approaches used to prepare the latter vaccines. For example, methods commonly used to inactivate bacterial pathogens for dead-cell vaccines, including but not limited to heat treatment, UV-irradiation, and chemical treatment, may deleteriously affect a vaccine's effectiveness by reducing the antigenicity of relevant immunological epitopes (Holt, Enright et al. 1990), (Melamed, Leitner et al. 1991), (Lauvau, Vijh et al. 2001). The presence of viable bacteriophage may also serve as an additional efficacy-enhancing factor, increasing the effectiveness of a phage lysate via their antibacterial effect on the Targeted Bacteria.

### Use of Recombinant Bacteriophage

In one embodiment of the invention, homologous recombination techniques are used to introduce sequences encoding alternative proteins, non-functional proteins, or non-coding sequences into the bacteriophage DNA sequence. Such techniques are useful to remove or “knock-out” undesired traits of the Deposited Bacteriophage, or alternatively to introduce different traits. In a particularly preferred embodiment of the invention, homologous recombination is used to “knock-out” ORFs encoding proteins that maybe involved in a lysogenic cycle of the bacteriophage.

In another embodiment of the invention, homologous recombination is used to add, or knock-out genes involved in burst size. For example, homologous recombination is used to introduce alternative bacteriophage genes which delay the burst event or increase the phage burst size.

In another embodiment of the invention, recombinant bacteriophage harboring reporter system(s) is generated for various practical applications. One example of possible application of such system is species identification/confirmation of Targeted Bacteria as bacterial diagnostics. Another possible application is the detection of the presence of viable cells of Targeted Bacteria to which the Deposited Bacteriophages have specificity. Following the techniques of Loessner et al., for example, one of skill in the art can generate recombinant reporter bacteriophage (Loessner, Rees et al. 1996). For example, the Vibrio harveyi luxAB gene may be introduced into the bacteriophage DNA sequence using techniques such as homologous recombination. An ideal target for the introduction of the luxAB gene is immediately downstream and in frame with an ORF encoding bacteriophage capsid protein, thereby creating a sequence encoding a fusion protein. The preferable location of introduction of the luxAB gene sequence is particularly before any sequence encoding a transcriptional terminator downstream of the ORF encoding a capsid protein. Other bacteriophage ORF sequences which may function as useful sources of luxAB gene-fusions include gene sequences encoding tail-sheath proteins, or any other late gene region sequences encoding phage head or tail proteins. The resulting recombinant bacteriophage may be used with methods of the invention to detect the presence of viable cells of Targeted Bacteria.

In addition to the Vibrio harveyi luxAB gene, other reporter genes which are useful for the generation of reporter bacteriophage include, but are not limited to, the firefly luciferase gene.

The invention further contemplates the introduction of one or more of the above-described recombinant events. For example, a recombinant bacteriophage of the invention may harbor one or more reporter gene(s) as well as lack one or more genes associated with certain undesirable biological functions of the bacteriophage.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The invention may be practiced in ways other than those particularly described in the foregoing description and examples. The teachings provided herein of the invention can be applied to other purposes, other than the examples described below.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### EXAMPLES

The invention will be described below on the basis of special embodiments, which, however, are in no way to be taken to mean a restriction of the general inventive concept. These examples and methods are specific embodiments; however, the present invention is not limited to these examples and methods. It is known to the person skilled in the art that the invention can be carried out in the same manner by modifying the examples and methods described and/or by replacing individual examples or methods or parts of examples or methods by alternative examples or methods or alternative parts of examples or methods.

### Example 1—Deposited Bacteriophages Isolation

ECML-1, ECML-10, ECML-123-2, ECML183-2, ECML-359, ECML-363, ECML-606-1, ECCR-664-1 bacteriophages were isolated from various environmental sources using lysis of the Targeted Bacteria to form plaques in bacterial lawns as a means of detecting the presence of bacteriophage having lytic specificity for the Targeted Bacteria. Plaques were harvested, diluted, and re-plated on bacterial lawns through a process of serial enrichment until a single bacteriophage species, or monophage, was obtained. This process allowed for selection of highly specific, lytic bacteriophage. The isolates obtained using the technique recited supra may be cultured using the techniques as set forth herein. The bacteriophage was deposited with the ATCC. The ATCC Deposit No. of the bacteriophages are shown in parenthesis adjacent to the bacteriophage: ECML-1 (PTA-127245), ECML-10 (PTA-127246), ECML-123-2 (PTA-121408), ECML183-2 (PTA-127247), ECML-359 (PTA-121406), ECML-363 (PTA-121407), ECML-606-1 (PTA-127248), ECCR-664-1 (PTA-127249).

### Example 2—Deposited Bacteriophages Concentration Determination

Concentration of the Deposited Bacteriophages may be determined using techniques known in the art (Adams 1959). When a single phage particle encounters a permissive bacterium, it will lyse it with the concomitant release of newly formed phage particles. When phages are mixed with host cells and poured in a layer of soft agar on the surface of a nutrient agar plate supporting bacterial growth, the cells will resume growth. In areas where no phages are present the bacteria will grow to stationary phase, forming a smooth opaque layer or lawn in the overlay. In areas where phages are present, phage progeny from each infected bacterium will infect neighboring bacteria, resulting in a growing zone of lysis full of liberated phage particles which eventually becomes visible to the naked eye as a plaque in the otherwise smooth bacterial lawn.

These plaques can be counted, and their number is commonly used for expressing phage titer in plaque-forming units or PFU. Using this approach, concentration of the Deposited Bacteriophages may be determined. Briefly: (1) Various dilutions of the Deposited Bacteriophages preparation are prepared; for example, by mixing 0.1 ml of phage sample with 9.9 ml of sterile Mueller Hinton broth. The samples are gently but thoroughly mixed. 0.5 ml of this mixture (which is a 10⁻² dilution of the original sample) is mixed with 4.5 ml of sterile Mueller Hinton broth (10⁻³ dilution). Several 10-fold dilutions are prepared in a similar fashion; (2) the contents of the tubes (1 ml of various dilutions) are transferred into sterile 10 ml culture tubes and 0.1 ml of host bacterial culture are added. The sample is mixed gently before proceeding immediately to the next step; (3) 3-5 ml of warm (45-50° C.) 0.7% LB agar (top agar) are added. The sample is mixed quickly and very gently. Then, the entire contents of the culture tube are poured onto a plate containing solidified LB—or technically equivalent—agar (bottom agar). The plates are slid in circles a few times on the bench top immediately after pouring; (4) after sitting at room temperature for 10 min to allow the top agar to harden, the plates are inverted and placed into a 37° C. incubator and incubated overnight; (5) the next morning, the number of plaques on the plate with 30-300 individual well-spaced plaques are counted and the titer calculated and expressed as PFU/mL of the starting sample.

### Example 3—Production of the Deposited Bacteriophages

The Deposited Bacteriophages are produced using a culture system. More specifically, strain of the host Targeted Bacteria or other closely related bacterial species on which the bacteriophage can propagate is cultured in batch culture, followed by inoculation of the bacteriophage at the pre-determined multiplicity of infection (MOI; ratio of phage vs. host bacteria). Following incubation and bacterial lysis, the bacteriophage is harvested and purified and/or concentrated to yield phage progeny suitable for the uses enumerated herein. Purification and concentration procedures included variously processing through filtration system(s), centrifugation (including continuous-flow centrifugation) or other known bacteriophage purification and concentration techniques (Adams 1959). The deposited bacteriophages produced as described above could be used as aqueous solution, or further processed to remove moisture and create a dry powder formulation, e.g., in the presence of a suitable excipient such as, for example, maltodextrin.

The invention provides compositions comprising active viral particles of the bacteriophage capable of lysing strains of Targeted Bacteria. The concentration of bacteriophage is determined using phage titration protocols as outlined in Example 2. The final concentration of the bacteriophage is adjusted by concentration, if a more concentrated phage composition is desired, via filtration, centrifugation, or other means, or by dilution, if a less concentrated phage composition is desired, with water or buffer to yield a phage titer of 10⁶ to 10¹² PFU/mL, preferably 10⁹ to 10¹² PFU/mL. The resulting bacteriophage compositions are generally stored at 2-8° C. in dark; alternatively, preparations can be freeze-dried or spray-dried for storage, or can be encapsulated and stabilized with protein, lipid, polysaccharide, or mixtures thereof. Upon reconstitution, the phage titer can be verified using phage titration protocols and host bacteria. One of skill in the art is capable of determining bacteriophage titers using widely known bacteriophage assay techniques (Adams 1959).

### Example 4—Application of the Deposited Bacteriophages for Preservation of Food Products

The bacteriophage produced using the methods of the present invention may be dispersed in an appropriate aqueous solution or dried powder and applied to the surface of food products. Alternatively, the bacteriophage may be included with a cheese culture or other microbially active foodstuff prior to or during processing.

### Example 5—Application of the Deposited Bacteriophages for Reducing the Risk of Foodborne Illness

The bacteriophage produced using the methods of the present invention may be given to animals (including humans) in an appropriate aqueous solution or enteric coated gel capsule to enhance gut resilience against pathogenic E. coli bacteria and reduce the severity or risk of development of, foodborne illness (including diarrhea, cramps, fever, and pain or other discomfort such as bloating) due to consumption of foods that may be contaminated with pathogenic E. coli.

### Example 6 Isolation of the Bacteriophage DNA

Bacteriophage DNA, a derivative of the bacteriophage, can be used for various applications such as for preparing DNA-based vaccines as well as for analytical purposes, e.g., for identifying the bacteriophage such as genome sequencing or RFLP profile determination and comparison. For example, phage DNA can be isolated using the standard phenol extraction technique, or a suitable commercial kit such as the Qiagen Plasmid Miniprep kit (Valencia, CA) or the Lambda Mini Kit (Qiagen, Inc.; Valencia, CA).

### Example 7—Restriction Fragment Length Polymorphism (RFLP) Profile

RFLP can be used to identify the Deposited Bacteriophages or its progeny. The progeny will have a substantially equivalent RFLP DNA profile as the RFLP DNA profile of the original bacteriophage, as defined by (Tenover, Arbeit et al. 1995). A reference RFLP profile of the Deposited Bacteriophages are shown in FIG. 1. DNA was isolated from the bacteriophage using Qiagen Plasmid Miniprep or Midiprep kits (Valencia, CA) according to the manufacturer's directions. The DNA can be quantitated by measuring absorbance at 260 nm. Approximately 0.5-1 mg of DNA could be digested with an appropriate restriction enzyme, and RFLP profile determined on an agarose gel after staining with ethidium bromide. RFLP patterns of the Deposited Bacteriophages after EcoRV digest are shown in FIG. 1.

### Example 8—Genome Analysis and Average Nucleotide Identity of the E. coli Phages

Full genome sequencing and sequence analysis could be used to identify the Deposited Bacteriophages or its progeny. The progeny will have ANI≥95% to be considered “Same Species” (including “Substantially Equivalent” to the Deposited Bacteriophages, or its “Progeny” or “Derivative”) as defined by Olm (Olm 2017) and Jain et al. (Jain, Rodriguez et al. 2018). All Deposited Bacteriophages have been sequenced. The GenBank accession numbers and other pertinent (genomic) information for the Deposited Bacteriophages are given in Table 2.

FIG. 2 shows a Force Graph activity visualization of the Deposited Phages against 738 E. coli strains, wherein the Force Graph was created using Intralytix's proprietary PhageSelector™ program. The green nodes represent the eight Deposited Bacteriophages. The blue nodes are collections of E. coli strains. Lines connecting green nodes to blue nodes indicate a Deposited Bacteriophage which can lyse the strains belonging to a node at a given concentration. Blue nodes that are larger in size and cluster towards the center of the figure denote that a larger number of strains are being lysed by multiple phages (i.e., redundant lytic spectrum coverage); while smaller nodes which cluster near the edges of the figure show a smaller number of strains that are susceptible to fewer monophages.

### Example 9—Lytic Specificity of the Deposited Bacteriophages

Seven hundred thirty-eight (738) E. coli strains were examined for their susceptibility to each of the eight Deposited Bacteriophages by the drop-on-lawn method, also known as the “spot test” method, essentially as described by (Adams 1959). Summary of the testing results is presented in Table 7. Of the eight deposited bacteriophages, ECML-363 has the broadest target range when tested against the entire collection of 738 E. coli strains (kills about 58% of all strains), while ECML-606-1 only targets about 6% of these strains (Table 7). Noteworthy, these data are for all E. coli strains in the Intralytix's bacterial strain collection, including both pathogenic and non-pathogenic E. coli. The efficacy of the Deposited Bacteriophages is considerably better against pathogenic E. coli. Just to give a few examples: (1) ECML-1 phage alone kills 100% of the eight K88 pathogenic E. coli in this collection; (2) Two of the Deposited Phages (ECCR-664-1 and ECML-183-2) combined kill 96% of 214 pathogenic STEC strains (Table 8).

In Tables 7 and 8 below, “Unique” means a number of unique kills by a particular phage (e.g., that the particular Deposited Phage has killed E. coli strains not killed by another phage). In Tables 7 and 8 below, “Kills” means a number of targeted strains of E. coli that were killed.

TABLE 7
Activity of the Deposited Bacteriophages against 738 E. coli strains

| Rank | Phage | Kills | Resistant | Unique | Cocktail* |
|---|---|---|---|---|---|
| 1 | ECML-363 | 426 | 283 | 426 | 426 (58%) |
| 2 | ECML-1 | 212 | 469 | 107 | 533 (72%) |
| 3 | ECML-10 | 261 | 420 | 50 | 583 (79%) |
| 4 | ECML-359 | 409 | 301 | 32 | 615 (83%) |
| 5 | ECCR-664-1 | 82 | 45 | 14 | 629 (85%) |
| 6 | ECML-183-2 | 40 | 13 | 4 | 633 (86%) |
| 7 | ECML-123-2 | 242 | 458 | 2 | 635 (86%) |
| 8 | ECML-606-1 | 44 | 57 | 0 | 635 |

TABLE 8
Activity of the Deposited Bacteriophages
against 214 pathogenic STEC strains

| Rank | Phage | Kills | Resistant | Unique | Cocktail* |
|---|---|---|---|---|---|
| 1 | ECML-359 | 179 | 28 | 179 | 179 (84%) |
| 2 | ECCR-664-1 | 75 | 14 | 25 | 204 (95%) |
| 3 | ECML-183-2 | 32 | 10 | 3 | 207 (97%) |
| 4 | ECML-363 | 177 | 30 | 1 | 208 (97%) |
| 5 | ECML-1 | 10 | 173 | 0 | 209 (98%) |
| 6 | ECML-10 | 69 | 114 | 0 | 209 |
| 7 | ECML-123-2 | 46 | 155 | 0 | 209 |
| 8 | ECML-606-1 | 6 | 37 | 0 | 209 |

*Cocktail - mixture of two or more individual phages. Each row shows number of E. coli strains lysed by cocktail of phages containing all phages through that row inclusive. For example, in Table 8, line 3 (Rank 3) shows cocktail that contains 3 phages (ECML-359, ECCR-664-1, and ECML-183-2) lysing 207 of the 214 pathogenic STEC strains.

### Example 10—Detection of Targeted Bacteria in Various Samples

The bacteriophage or its derivative, such as lytic enzyme, produced using the methods of the present invention is used to specifically lyse Targeted Bacteria without affecting any other prokaryotic or eukaryotic cells that may be present in the sample; thus, specifically eliciting their release of measurable bacterial products such as AK or ATP. Briefly: (1) Samples to be analyzed are obtained and suspended in appropriate buffer, (2) The Deposited Bacteriophages are added to the suspensions, as a result of which the Targeted Bacteria cells present in the samples are lysed and their ATP is released, (3) A luciferin+luciferase preparation is added to the mixtures, and (5) The mixtures' luminescence is measured within 60 sec, and the results are displayed on a handheld luminometer. It may be possible to establish a correlation between the luminometer readings and the number of Targeted Bacteria cells lysed (in general, the average amount of ATP per bacterial cell is 0.5-1.0 fg; precise correlation between the luminometer readings and the number of Targeted Bacteria cells should be experimentally established). If Targeted Bacteria cells are not present in the food samples analyzed, bacterial lysis and ATP-release will not occur. Detection of pathogenic E. coli strain in a various sample (including food samples, environmental samples, and clinical specimens) employing the deposited bacteriophages or derivatives thereof, as described above, for example, is also contemplated by this invention.

### Example 11—Preparing Vaccines and Bacterins

One example of utilizing bacteriophages to prepare vaccines and bacterins is to use the lytic Deposited Bacteriophages to lyse specific strains of the Targeted Bacteria, which will yield bacterial lysates containing minimally affected immunological epitopes of the bacteria. The phage may be removed from the final vaccine/bacterin preparation. Alternatively, it may be retained unaltered in the preparation because its lytic activity against Targeted Bacteria that may be present in the mammalian organism being vaccinated may increase the preparation's efficacy. In one preferred embodiment of the present invention: (i) the most prevalent, problematic strains of the Targeted Bacteria are chosen so that the vaccine/bacterin contains the immunological epitopes that are most relevant for protecting against the infection, and (ii) the bacteriophage is kept unaltered in the final vaccine/bacterin, at levels ranging from 10⁶-10¹² PFU/mL.

Bacteriophage-based vaccines and bacterins also may be prepared by using derivatives of the Deposited Bacteriophages to lyse the Targeted Bacteria. An example of the general methodology can be briefly outlined from a study of a Helicobacter pylori bacterin (Panthel, Jechlinger et al. 2003). The authors used E. coli-H. pylori shuttle plasmid pHel2 and lysis gene e of bacteriophage (pX174 to construct H. pylori lysis plasmid pHPC38, which they introduced into H. pylori strain P79. At a pre-determined time, the authors triggered e gene-expression to elicit bacterial lysis, and they found that the lysate/bacterin protected BALB/c mice against H. pylori infection. Vaccination using a vaccine comprising bacterins produced by lysis of pathogenic E. coli species or strain(s) employing the deposited bacteriophages or derivatives thereof is also contemplated by this invention. Production of the vaccine as well as vaccination using such a vaccine may be performed by methods known to a person of ordinary skill in the art.

### Example 12—Application of the Deposited Bacteriophages for Reducing the Risk of, Mitigating, or Treating Gastrointestinal Diseases Caused by Pathogenic E. coli in Humans

One or more of the Deposited Bacteriophage may be included in a pharmaceutical formulation that could be given to human either at risk of exposure to pathogenic E. coli and before the onset of clinical symptoms (i.e., reducing the risk of disease) or after they develop clinical symptoms—e.g., diarrhea—that are associated with pathogenic E. coli infections (i.e., treatment of disease). Such pharmaceutical formulation maybe administered orally to humans, with the oral dosage form formulated such that the Deposited Bacteriophage(s) are released into the intestine after passing through the stomach, to protect phage from the acidic environment (typical pH of 1.5 to 3.5) of the stomach. One example of such formulation would be enteric coated gel caps, preferably in size “0” or “00,” with the capsule capacity of 408-816 mg or 546-1,092 mg, respectively.

One or more of the Deposited Bacteriophage may be embedded in nanosized polymeric microgel particles including a cross-linked polymer network of polyionic segments and neutral segments as described in Vinogradov (Vinogradov 2006). The microgel particles will have diameters of about 1 μm to about 4 μm, suitable sized for phagocytosis by macrophages. Preferably, non-mammalian carbohydrates such as mannose, chitosan, and β-glucan may be incorporated into the microgel particles to induce phagocytosis of the microgel particles by macrophages. The microgel particles may be given to animals (including humans) to enhance resilience against pathogenic E. coli bacteria and to reduce the risk and/or severity of illness. For example, the phage-containing microgel particles may be given to humans by oral, intravenous, or intradermal or intraparenchymal injection of an appropriate phage-containing microgel preparation to reduce the risk of, mitigate, and/or treat pathogenic E. coli infections caused by consumption of foods or drinking water that may be contaminated with pathogenic E. coli.

### Example 13—Application of the Deposited Bacteriophages for Reducing the Risk of, Mitigating, or Treating UTI Infections Caused by Pathogenic E. coli in Humans

One or more of the Deposited Bacteriophage may be given to humans orally to reduce the risk of, prevent, or treat UTI caused by pathogenic E. coli, including UPEC. Alternative treatment regimens may include administering one or more of the Deposited Bacteriophages to humans by intravenous (i.v.) injection, intramuscular (i.m.) injection, intraperitoneal (i.p.) injection, via irrigation of the urinary tract, and/or by using suprapubic trocar and/or transurethral catheters, or by any combination of these approaches.

### Example 14—Application of the Deposited Bacteriophages for Reducing the Risk of, Mitigating, or Treating Diseases Caused by Pathogenic E. coli in Non-Human Animals

One or more of the Deposited Bacteriophage may be given to non-human animals to enhance resilience against pathogenic E. coli bacteria and to reduce the risk and/or severity of illness. For example, phage preparation containing one or more of the deposited bacteriophages can be given to one or more food animals (any species commonly recognized as livestock including, but not limited to, poultry, cattle, swine, and sheep) to either reduce the risk of, mitigate, or treat respiratory and systemic diseases caused by pathogenic E. coli, including aerosacculitis, perihepatitis, polyserositis, pericarditis, egg peritonitis, salphingitis, coligranuloma, omphalitis, cellulitis, and osteomyelitis/arthritis, septicemia and (diseases commonly referred as avian colibacillosis). In this one example, live poultry includes chickens, turkeys, ducks, and other avian species. Also, in this example, pathogenic E. coli are various APEC serotypes, including O1, O2 (including O2:K2, which is one of the most common serotypes among APEC worldwide), and O78 (and especially O78:K80) which accounts for more than 80% of the illness (Dho-Moulin and Fairbrother 1999), (Rodriguez-Siek, Giddings et al. 2005), (Ebrahimi-Nik, Bassami et al. 2018). In one embodiment, one or more of the Deposited Bacteriophage produced according to paragraph may be given to live poultry by mixing with their drinking water or feed, to reduce the risk of APEC-associated illness or to treat colibacillosis.

### PUBLICATIONS, PATENTS AND PATENT APPLICATIONS

All publications, patents and patent applications are herein incorporated by reference in their entirety to the same extent as if each individual publication, patent or patent application was specifically and individually indicated to be incorporated by reference in its entirety.

Those skilled in the art will recognize or be able to ascertain using no more than routine experimentation, many equivalents to the specific embodiments of the invention described herein. Such equivalents are intended to be encompassed by the following claims.

### REFERENCES

ADDIN EN.REFLIST Abuladze, T., M. Li, M. Y. Menetrez, T. Dean, A. Senecal and A. Sulakvelidze (2008). “Bacteriophages reduce experimental contamination of hard surfaces, tomato, spinach, broccoli, and ground beef by Escherichia coli O157:H7.” Appl Environ Microbiol 74(20): 6230-6238.
Adams, M. H. (1959). Enumeration of bacteriophage particles. Bacteriophages. London, Interscience Publishers, Ltd.: 27-34.
Adams, M. H. (1959). Methods of study bacterial viruses. Bacteriophages. London, Interscience Publishers, Ltd.: 443-522.
Alisky, J., K. Iczkowski, A. Rapoport and N. Troitsky (1998). “Bacteriophages show promise as antimicrobial agents.” J Infect 36(1): 5-15.
Aymerich, T., P. A. Picouet and J. M. Monfort (2008). “Decontamination technologies for meat products.” Meat Sci 78(1-2): 114-129.
Behrsing, J., S. Winkler, P. Franz and R. Premier (2000). “Efficacy of chlorine for inactivation of Escherichia coli on vegetables.” Postharvest Biol Technol 19: 187-192.
Belanger, L., A. Garenaux, J. Harel, M. Boulianne, E. Nadeau and C. M. Dozois (2011). “Escherichia coli from animal reservoirs as a potential source of human extraintestinal pathogenic E. coli.” FEMS Immunol Med Microbiol 62(1): 1-10.
Bell, B. P., M. Goldoft, P. M. Griffin, M. A. Davis, D. C. Gordon, P. I. Tarr, C. A. Bartleson, J. H. Lewis, T. J. Barrett, J. G. Wells and et al. (1994). “A multistate outbreak of Escherichia coli O157:H7-associated bloody diarrhea and hemolytic uremic syndrome from hamburgers. The Washington experience.” JAMA 272(17): 1349-1353.
Beutin, L. (1999). “Escherichia coli as a pathogen in dogs and cats.” Vet Res 30(2-3): 285-298.
Buzby, J. C. (2001). “Children and microbial foodborne illness.” Food Review 24: 32-37.
Buzby, J. C. and T. Roberts (1997). “Economic costs and trade impacts of microbial foodborne illness.” World Health Stat Q 50(1-2): 57-66.
Buzby, J. C. and T. Roberts (2009). “The economics of enteric infections: human foodborne disease costs.” Gastroenterology 136(6): 1851-1862.
Carter, C. D., A. Parks, T. Abuladze, M. Li, J. Woolston, J. Magnone, A. Senecal, A. M. Kropinski and A. Sulakvelidze (2012). “Bacteriophage cocktail significantly reduces Escherichia coli O157: H7 contamination of lettuce and beef, but does not protect against recontamination.” Bacteriophage 2(3): 178-185.
Davidson, P. M. and M. A. Harrison (2002). “Resistance and adaptation to food antimicrobials, sanitizers, and other process controls.” Food Technology 56(11): 69-78.
Dho-Moulin, M. and J. M. Fairbrother (1999). “Avian pathogenic Escherichia coli (APEC).” Vet Res 30(2-3): 299-316.
Dziva, F. and M. P. Stevens (2008). “Colibacillosis in poultry: unravelling the molecular basis of virulence of avian pathogenic Escherichia coli in their natural hosts.” Avian Pathol 37(4): 355-366.
Ebrahimi-Nik, H., M. R. Bassami, M. Mohri, M. Rad and M. I. Khan (2018). “Bacterial ghost of avian pathogenic E. coli (APEC) serotype O78:K80 as a homologous vaccine against avian colibacillosis.” PLoS One 13(3): e0194888.
Etcheverría, A. I., P. M. A. Lucchesi, A. Kruger, A. B. Bentancor and N. L. Padola (2016). Escherichia coli in animals. Escherichia coli in the Americas. A. Torres, Springer, Cham. Fairbrother, J. M., E. Nadeau and C. L. Gyles (2005). “Escherichia coli in postweaning diarrhea in pigs: an update on bacterial types, pathogenesis, and prevention strategies.” Anim Health Res Rev 6(1): 17-39.
Frenzen, P. D., A. Drake, F. J. Angulo and G. Emerging Infections Program FoodNet Working (2005). “Economic cost of illness due to Escherichia coli O157 infections in the United States.” J Food Prot 68(12): 2623-2630.
Ghunaim, H., M. A. Abu-Madi and S. Kariyawasam (2014). “Advances in vaccination against avian pathogenic Escherichia coli respiratory disease: potentials and limitations.” Vet Microbiol 172(1-2): 13-22.
Greig, D. J., F. M. Gulland, W. A. Smith, P. A. Conrad, C. L. Field, M. Fleetwood, J. T. Harvey, H. S. Ip, S. Jang, A. Packham, E. Wheeler and A. J. Hall (2014). “Surveillance for zoonotic and selected pathogens in harbor seals Phoca vitulina from central California.” Dis Aquat Organ 111(2): 93-106.
Guabiraba, R. and C. Schouler (2015). “Avian colibacillosis: still many black holes.” FEMS Microbiol Lett 362(15): fnv118.
Holt, M. E., M. R. Enright and T. J. Alexander (1990). “Immunisation of pigs with killed cultures of Streptococcus suis type 2.” Res Vet Sci 48(1): 23-27.
Jain, C., R. L. Rodriguez, A. M. Phillippy, K. T. Konstantinidis and S. Alum (2018). “High throughput ANI analysis of 90K prokaryotic genomes reveals clear species boundaries.” Nat Commun 9(1): 5114.
Kaper, J. B., J. P. Nataro and H. L. Mobley (2004). “Pathogenic Escherichia coli.” Nat Rev Microbiol 2(2): 123-140.
Kathayat, D., D. Lokesh, S. Ranjit and G. Rajashekara (2021). “Avian pathogenic Escherichia coli (APEC): an overview of virulence and pathogenesis factors, zoonotic potential, and control strategies.” Pathogens 10(4).
Kim, K. S. (2016). “Human meningitis-associated Escherichia coli.” EcoSal Plus 7(1).
Koren, S., B. P. Walenz, K. Berlin, J. R. Miller, N. H. Bergman and A. M. Phillippy (2017). “Canu: scalable and accurate long-read assembly via adaptive k-mer weighting and repeat separation.” Genome Res 27(5): 722-736.
Kutter, E. and A. Sulakvelidze (2005). Bacteriophages: Biology and Application. Boca Raton, FL, CRC Press.
Lauvau, G., S. Vijh, P. Kong, T. Horng, K. Kerksiek, N. Serbina, R. A. Tuma and E. G. Pamer (2001). “Priming of memory but not effector CD8 T cells by a killed bacterial vaccine.” Science 294(5547): 1735-1739.
Loessner, M. J., C. E. Rees, G. S. Stewart and S. Scherer (1996). “Construction of luciferase reporter bacteriophage A511::luxAB for rapid and sensitive detection of viable Listeria cells.” Appl Environ Microbiol 62(4): 1133-1140.
Luppi, A. (2017). “Swine enteric colibacillosis: diagnosis, therapy and antimicrobial resistance.” Porcine Health Management 3(1): 16.
McGrath, S. and D. van Sinderen (2007). Bacteriophage: Genetics and Molecular Biology, Caister Academic Press.
Melamed, D., G. Leitner and E. D. Heller (1991). “A vaccine against avian colibacillosis based on ultrasonic inactivation of Escherichia coli.” Avian Dis 35(1): 17-22.
Merril, C. R., B. Biswas, R. Carlton, N. C. Jensen, G. J. Creed, S. Zullo and S. Adhya (1996). “Long-circulating bacteriophage as antibacterial agents.” Proc Natl Acad Sci USA 93(8): 3188-3192.
Mokgatla, R. M., V. S. Brozel and P. A. Gouws (1998). “Isolation of Salmonella resistant to hypochlorous acid from a poultry abattoir.” Lett Appl Microbiol 27(6): 379-382.
Moye, Z. D., J. Woolston and A. Sulakvelidze (2018). “Bacteriophage Applications for Food Production and Processing.” Viruses 10(4).
Olm, M. (2017). Are these microbes the “same”? microBEnet: the microbiology of the Built Environment network: microbe.net/2017/2002/2015/are-these-microbes-the-same/.
Olm, M. R., C. T. Brown, B. Brooks and J. F. Banfield (2017). “dRep: a tool for fast and accurate genomic comparisons that enables improved genome recovery from metagenomes through de-replication.” ISME J 11(12): 2864-2868.
Panthel, K., W. Jechlinger, A. Matis, M. Rohde, M. Szostak, W. Lubitz and R. Haas (2003). “Generation of Helicobacter pylori ghosts by PhiX protein E-mediated inactivation and their evaluation as vaccine candidates.” Infect Immun 71(1): 109-116.
Pennington, H. (2010). “Escherichia coli O157.” Lancet 376(9750): 1428-1435.
Prudent, V., G. Demarre, E. Vazeille, M. Wery, N. Quenech'Du, A. Ravet, J. Dauverd-Girault, E. van Dijk, M. A. Bringer, M. Descrimes, N. Barnich, S. Rimsky, A. Morillon and O. Espeli (2021). “The Crohn's disease-related bacterial strain LF82 assembles biofilm-like communities to protect itself from phagolysosomal attack.” Commun Biol 4(1): 627.
Rangel, J. M., P. H. Sparling, C. Crowe, P. M. Griffin and D. L. Swerdlow (2005). “Epidemiology of Escherichia coli O157:H7 outbreaks, United States, 1982-2002.” Emerg Infect Dis 11(4): 603-609.
Rodriguez-Siek, K. E., C. W. Giddings, C. Doetkott, T. J. Johnson and L. K. Nolan (2005). “Characterizing the APEC pathotype.” Vet Res 36(2): 241-256.
Scallan, E., R. M. Hoekstra, F. J. Angulo, R. V. Tauxe, M. A. Widdowson, S. L. Roy, J. L. Jones and P. M. Griffin (2011). “Foodborne illness acquired in the United States—major pathogens.” Emerg Infect Dis 17(1): 7-15.

Simoes, R. R., L. Poirel, P. M. Da Costa and P. Nordmann (2010). “Seagulls and beaches as reservoirs for multidrug-resistant Escherichia coli.” Emerg Infect Dis 16(1): 110-112.
Stenutz, R., A. Weintraub and G. Widmalm (2006). “The structures of Escherichia coli O-polysaccharide antigens.” FEMS Microbiol Rev 30(3): 382-403.
Sulakvelidze, A. (2011). “Safety by nature: potential bacteriophage applications.” Microbe 6(3): 122-126.
Sulakvelidze, A. and P. Barrow (2005). Phage therapy in animals and agribusiness. Bacteriophages: Biology and Applications. E. Kutter and A. Sulakvelidze. Boca Raton, FL, CRC Press: 335-380.
Sulakvelidze, A. and E. Kutter (2005). Bacteriophage therapy in humans. Bacteriophages: Biology and Application. E. Kutter and A. Sulakvelidze. Boca Raton, FL, CRC Press: 381-436.
Summers, W. C. (1999). Félix d'Herelle and the origins of molecular biology. New Haven, CT, Yale Univ Press.
Suttle, C. A. (2005). “Viruses in the sea.” Nature 437(7057): 356-361.
Suttle, C. A. (2007). “Marine viruses—major players in the global ecosystem.” Nat Rev Microbiol 5(10): 801-812.
Tenover, F. C., R. D. Arbeit, R. V. Goering, P. A. Mickelsen, B. E. Murray, D. H. Persing and B. Swaminathan (1995). “Interpreting chromosomal DNA restriction patterns produced by pulsed-field gel electrophoresis: criteria for bacterial strain typing.” J Clin Microbiol 33(9): 2233-2239.
Terlizzi, M. E., G. Gribaudo and M. E. Maffei (2017). “UroPathogenic Escherichia coli (UPEC) infections: virulence factors, bladder responses, antibiotic, and non-antibiotic antimicrobial strategies.” Front Microbiol 8: 1566.
van der Mee-Marquet, N., M. Loessner and A. Audurier (1997). “Evaluation of seven experimental phages for inclusion in the international phage set for the epidemiological typing of Listeria monocytogenes.” Appl Environ Microbiol 63(9): 3374-3377.
van Elk, C. E., M. W. van Dep Bildt, B. E. Martina, A. D. Osterhaus and T. Kuiken (2007). “Escherichia coli septicemia associated with lack of maternally acquired immunity in a bottlenose dolphin calf.” Vet Pathol 44(1): 88-92.
Vinogradov, S. V. (2006). “Colloidal microgels in drug delivery applications.” Curr Pharm Des 12(36): 4703-4712.
Walker, B. J., T. Abeel, T. Shea, M. Priest, A. Abouelliel, S. Sakthikumar, C. A. Cuomo, Q. Zeng, J. Wortman, S. K. Young and A. M. Earl (2014). “Pilon: an integrated tool for comprehensive microbial variant detection and genome assembly improvement.” PLoS One 9(11): e112963.
Wheeler, T. L., S. D. Shackelford and M. Koohmaraie (1999). “Trained sensory panel and consumer evaluation of the effects of gamma irradiation on palatability of vacuum-packaged frozen ground beef patties.” J. Anim Sci. 77(12): 3219-3224.
Yoon, S. H., S. M. Ha, J. Lim, S. Kwon and J. Chun (2017). “A large-scale evaluation of algorithms to calculate average nucleotide identity.” Antonie Van Leeuwenhoek 110(10): 1281-1286.

What is claimed is:

1. An isolated bacteriophage ECML-1 deposited under ATCC Deposit Accession No. PTA-127245, ECML-10 deposited under ATCC Deposit Accession No. PTA-127246, ECML-123-2 deposited under ATCC Deposit Accession No. PTA-121408, ECML-183-2 deposited under ATCC Deposit Accession No. PTA-127247, ECML-359 deposited under ATCC Deposit Accession No. PTA-121406, ECML-363 deposited under ATCC Deposit Accession No. PTA-121407, ECML-606-1 deposited under ATCC Deposit Accession No. PTA-127248, ECCR-664-1 deposited under ATCC Deposit Accession No. PTA-127249, or a combination thereof, said bacteriophages having lytic activity against pathogenic E. coli species strains.

2. A method for the reduction of food contamination and/or the incidence of food borne illnesses caused by pathogenic E. coli strains comprising contacting a food product or products with a microbial growth inhibiting effective amount of a bacteriophage composition comprising at least one of the isolated bacteriophage of claim 1 and variants thereof, wherein said variants have an average nucleotide identity over genome of ≥80% relative to the bacteriophage and retain the phenotypic characteristics of said bacteriophage, and wherein said bacteriophage and variants thereof have lytic activity against pathogenic E. coli strains.

3. The method according to claim 2, wherein the contact comprises spraying or misting or fogging the bacteriophage composition on the food product(s), by dipping or soaking or vacuum sealing the food product(s) in a solution containing a concentration of the bacteriophage composition sufficiently high to inhibit the growth of pathogenic E. coli strains, or adding, injecting or inserting the bacteriophage composition in said concentration into the food product(s).

4. A method for the reduction of food contamination and/or the incidence of food borne illnesses caused by pathogenic E. coli comprising contacting food processing equipment with a microbial growth inhibiting effective amount of a bacteriophage composition comprising at least one of the isolated bacteriophage of claim 1, and variants thereof, wherein said variants have an average nucleotide identity over genome of ≥80% relative to the bacteriophage and retain the phenotypic characteristics of said bacteriophage, and wherein said bacteriophage and variants thereof have lytic activity against pathogenic E. coli strains.

5. The method according to claim 4, wherein the contact comprises spraying or misting the bacteriophage composition on the food processing equipment, by dipping or soaking the food processing equipment in a solution containing a concentration of the bacteriophage composition sufficiently high to inhibit the growth of pathogenic E. coli strains, or adding, injecting or inserting the bacteriophage composition into the food processing equipment; or spraying or misting the bacteriophage composition on a surface used in food processing.

6. A method for the reduction of environmental contamination and/or the incidence of nosocomial infections caused by pathogenic E. coli comprising contacting porous and non-porous surfaces (including but not limited to floors, walls, beds, handheld remotes and other electronic equipment, and medical devises) in intensive care units, hospitals, urgent care centers, nursing homes, and other health care facilities with a microbial growth inhibiting effective amount of a bacteriophage composition comprising at least one of the isolated bacteriophage of claim 1 and variants thereof, wherein said variants have an average nucleotide identity over genome of ≥80% relative to the bacteriophage and retain the phenotypic characteristics of said bacteriophage, and wherein said bacteriophage and variants thereof have lytic activity against pathogenic E. coli strains.

7. A dietary supplement/feed additive/nutraceutical/pharmaceutical (drug) composition comprising one or more of an isolated bacteriophage of claim 1 and variants thereof, wherein said variants have an average nucleotide identity over genome of ≥80% relative to the bacteriophage and retain the phenotypic characteristics of said bacteriophage, and wherein said bacteriophage and variants thereof have lytic activity against pathogenic E. coli strains.

8. The dietary supplement/feed additive/nutraceutical/pharmaceutical (drug) composition according to any one of claim 7, wherein the composition is formulated as a capsule, gel capsule, enteric coated capsule, microgel, suppository, tablet, chewable composition, syrup, or gel.

9. The dietary supplement/feed additive/nutraceutical/pharmaceutical (drug) composition according to claim 7, wherein the composition further comprises a probiotic bacteria, preferably Lactobacillus species, preferably L. acidophilus, L. rhamnosus, L. gasseri, L. reuteri, L. bulgaricus, L. plantarum, L. johnsonii, L. paracasei, L. casei, L. salivarius, or L. lactis, Bifidobacterium species, preferably B. bifidum, B. longum, B. breve, B. infantis, B. lactis, or B. adolescentis, Streptococcus thermophilus, Bacillus cerus, Bacillus subtilis, Enterococcus faecalis, Enterococcus faecium, or a combination thereof.

10. The dietary supplement/feed additive/nutraceutical/pharmaceutical (drug) composition according to claim 7, wherein the composition further comprises a probiotic yeast, preferably Saccharomyces cerevisiae, Saccharomyces boulardii, Saccharomyces cerevisiae var. boulardii, Issatchenkia occidentalis, Lachancea thermotolerans, Metschnikowia ziziphicola, Torulaspora delbrueckii, or a combination thereof.

11. A method of reducing the risk thereof or mitigating infections caused by pathogenic E. coli comprising administering an effective amount of the dietary supplement/feed additive/nutraceutical/pharmaceutical (drug) composition according to claim 7.

12. A method of treating pathogenic E. coli-associated ailments in humans, such as kidney failure/hemolytic uremic syndrome, diarrhea (including hemorrhagic or very bloody diarrhea), thrombotic thrombocytopenic purpura (loss of blood platelets and kidney failure), comprising administering an effective amount of the dietary supplement/feed additive/nutraceutical/pharmaceutical (drug) composition according to claim 7.

13. A method of treating pathogenic E. coli-associated ailments in animals, such as bovine mastitis, pig colibacillosis, avian colibacillosis (including, but not limited to, aerosacculitis, perihepatitis, polyserositis, pericarditis, egg peritonitis, salphingitis, coligranuloma, omphalitis, cellulitis, and osteomyelitis/arthritis, septicemia and other diseases in chickens, turkeys, ducks, and other avian species), comprising administering an effective amount of the dietary supplement/feed additive/nutraceutical/pharmaceutical (drug) composition according to claim 7.

14. A method of increasing gut resilience against colonization with pathogenic E. coli bacteria in animals (including humans) comprising administering an effective amount of the dietary supplement/feed additive/nutraceutical/pharmaceutical (drug) composition according to claim 7.

15. A method of relieving gut discomfort including but not limited to diarrhea, cramps, fever and pain in animals (including humans) by reducing, or eliminating colonization with pathogenic E. coli bacteria comprising administering an effective amount of the dietary supplement/feed additive/nutraceutical/pharmaceutical (drug) composition according to claim 7.

16. A method for modulating an animal's (including human's) microbiome by preventing or reducing colonization by pathogenic E. coli bacteria strains of a subject comprising administration of an effective amount of a composition comprising at least one of the isolated bacteriophage of claim 1 and variants thereof, wherein said variants have an average nucleotide identity over genome of ≥80% relative to the bacteriophage and retain the phenotypic characteristics of said bacteriophage, and wherein said bacteriophage and variants thereof have lytic activity against pathogenic E. coli strains.

17. The method according to claim 16, wherein the pathogenic E. coli strain is Shiga-toxin producing E. coli (STEC), enterohemorrhagic E. coli (EHEC), enteropathogenic E. coli (EPEC), enterotoxigenic E. coli (ETEC), enteroaggregative E. coli (EAEC), enteroinvasive E. coli (EIEC) diffusely adherent E. coli (DAEC), uropathogenic E. coli (UPEC), meningitis-associated E. coli (MNEC), avian pathogenic E. coli (APEC), sepsis-associated E. coli, (SAEC), mammary pathogenic E. coli, endometrial pathogenic E. coli, adherent invasive E. coli (AIEC), necrotoxigenic E. coli, or a combination thereof.

18. The method according to claim 16, wherein the bacteriophage is present in the composition in an amount of 10⁶ and 10¹¹ PFU.

19. A method for vaccinating a subject against pathogenic E. coli bacteria, the method comprising administering to the subject a composition comprising a bacterial lysate obtained by lysing the pathogenic E. coli strain with a bacteriophage or an isolated progeny thereof according to claim 1.

20. A method for detecting a pathogenic E. coli in a food or environmental or clinical sample, the method comprising treating the sample with a bacteriophage or an isolated progeny or derivative thereof according to claim 1, thereby specifically eliciting release of measurable bacterial products, and measuring the released bacterial product.
